SEQUENCE LISTING (25 sequences)

SEQ ID NO: 1 - protein, 126 aa, Homo sapiens [sequence omitted]

SEQ ID NO: 2 - protein, 134 aa, Homo sapiens
  REGION 1..134: note = extracellular domain of human CD28
NKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKP

SEQ ID NO: 3 - protein, 42 aa, Homo sapiens
  REGION 1..42: note = intracellular domain of 4-1BB
KRGRKKLLYIFKQPFMRPVQTTQEEDGCSCRFPEEEEGGCEL

SEQ ID NO: 4 - protein, 237 aa, Homo sapiens
  REGION 1..237: note = aCD80(scFv), clone hu1F1
QLVQSGAEVKKPGASVKVSCKPSGFNIKDYYMHWVRQAPGQGLEWIGWIDPENGNTLYDPKFQGKATITADTSTSTAYMELSSLRSEDTAVYYCAREGLFFAYWGQGTLVTVSSGGGGSGGGGSGGGGSDIQMTQSPSSLSASVGDRVTITCSVSSSISSSNLHWYQQKPGKAPKPLIYGTSNLASGVPSRFSGSGSGTDYTLTISSLQPEDVATYYCQQWSSYPLTFGQGTKVEIK

SEQ ID NO: 5 - protein, 243 aa, Homo sapiens
  REGION 1..243: note = aCD86(scFv), clone hu3D1
QVQLVQSGAEVKKPGSSVKVSCKASGYTFTDYAIQWVRQAPGQGLEWIGVINIYYDNTNYNQKFKGKATMTVDKSTSTAYMELSSLRSEDTAVYYCARAAWYMDYWGQGTLVTVSSGGGGSGGGGSGGGGSDIVLTQSPDSLAVSLGERATISCKSSQSLLNSRTRENYLAWYQQKPGQPPKLLIYWASTRESGVPDRFSGSGSGTDFTLTISSLQAEDVAVYYCTQSYNLYTFGQGTKVEIK

SEQ ID NO: 6 - protein, 500 aa, synthetic construct
  REGION 1..500: note = fused aCD80(scFv)-aCD86(scFv) binding domain
QLVQSGAEVKKPGASVKVSCKPSGFNIKDYYMHWVRQAPGQGLEWIGWIDPENGNTLYDPKFQGKATITADTSTSTAYMELSSLRSEDTAVYYCAREGLFFAYWGQGTLVTVSSGGGGSGGGGSGGGGSDIQMTQSPSSLSASVGDRVTITCSVSSSISSSNLHWYQQKPGKAPKPLIYGTSNLASGVPSRFSGSGSGTDYTLTISSLQPEDVATYYCQQWSSYPLTFGQGTKVEIKGGGGSGGGGSGGGGSGGGGSQVQLVQSGAEVKKPGSSVKVSCKASGYTFTDYAIQWVRQAPGQGLEWIGVINIYYDNTNYNQKFKGKATMTVDKSTSTAYMELSSLRSEDTAVYYCARAAWYMDYWGQGTLVTVSSGGGGSGGGGSGGGGSDIVLTQSPDSLAVSLGERATISCKSSQSLLNSRTRENYLAWYQQKPGQPPKLLIYWASTRESGVPDRFSGSGSGTDFTLTISSLQAEDVAVYYCTQSYNLYTFGQGTKVEIK

SEQ ID NO: 7 - skipped

SEQ ID NO: 8 - protein, 9 aa, synthetic construct
  REGION 1..9: note = ITAM motif (YX1X2L/I)(X3)n(YX1X2L/I)
  SITE 2..3: note = The amino acids at positions 2 and 3 are independently any amino acid
  VARIANT 4: note = The amino acid at position 4 may also be Ile
  SITE 5: note = X is a cluster of n amino acids, where n is 6, 7, or 8, and each of the 6-8 amino acids is any amino acid
  SITE 7..8: note = The amino acids at positions 7 and 8 are independently any amino acid
  VARIANT 9: note = The amino acid at position 9 may also be Ile
YXXLXYXXL

SEQ ID NO: 9 - protein, 189 aa, synthetic construct
  REGION 1..189: note = CTLA-4(ec+tm)-4-1BB (ic) CCR
KAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTKRGRKKLLYIFKQPFMRPVQTTQEEDGCSCRFPEEEEGGCEL

SEQ ID NO: 10 - protein, 203 aa, synthetic construct
  REGION 1..203: note = CD28(ec+tm)-4-1BB(ic) CCR
NKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCEL

SEQ ID NO: 11 - protein, 345 aa, synthetic construct
  REGION 1..345: note = aCD80(scFv)-CD28(ec+tm)-4-1BB(ic) CCR
QLVQSGAEVKKPGASVKVSCKPSGFNIKDYYMHWVRQAPGQGLEWIGWIDPENGNTLYDPKFQGKATITADTSTSTAYMELSSLRSEDTAVYYCAREGLFFAYWGQGTLVTVSSGGGGSGGGGSGGGGSDIQMTQSPSSLSASVGDRVTITCSVSSSISSSNLHWYQQKPGKAPKPLIYGTSNLASGVPSRFSGSGSGTDYTLTISSLQPEDVATYYCQQWSSYPLTFGQGTKVEIKIEVMYPPPYLDNERSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCEL

SEQ ID NO: 12 - protein, 350 aa, synthetic construct
  REGION 1..350: note = aCD86(scFv)-CD28(ec+tm)-4-1BB(ic) CCR
QVQLVQSGAEVKKPGSSVKVSCKASGYTFTDYAIQWVRQAPGQGLEWIGVINIYYDNTNYNQKFKGKATMTVDKSTSTAYMELSSLRSEDTAVYYCARAAWYMDYWGQGTLVTVSSGGGGSGGGGSGGGGSDIVLTQSPDSLAVSLGERATISCKSSQSLLNSRTRENYLAWYQQKPGQPPKLLIYWASTRESGVPDRFSGSGSGTDFTLTISSLQAEDVAVYYCTQSYNLYTFGQGTKVEIKEVMYPPPYLDNERSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCEL

SEQ ID NO: 13 - protein, 607 aa, synthetic construct
  REGION 1..607: note = aCD80(scFv)-aCD86(scFv)-CD28(ec+tm)-4-1BB(ic) CCR
QLVQSGAEVKKPGASVKVSCKPSGFNIKDYYMHWVRQAPGQGLEWIGWIDPENGNTLYDPKFQGKATITADTSTSTAYMELSSLRSEDTAVYYCAREGLFFAYWGQGTLVTVSSGGGGSGGGGSGGGGSDIQMTQSPSSLSASVGDRVTITCSVSSSISSSNLHWYQQKPGKAPKPLIYGTSNLASGVPSRFSGSGSGTDYTLTISSLQPEDVATYYCQQWSSYPLTFGQGTKVEIKGGGGSGGGGSGGGGSGGGGSQVQLVQSGAEVKKPGSSVKVSCKASGYTFTDYAIQWVRQAPGQGLEWIGVINIYYDNTNYNQKFKGKATMTVDKSTSTAYMELSSLRSEDTAVYYCARAAWYMDYWGQGTLVTVSSGGGGSGGGGSGGGGSDIVLTQSPDSLAVSLGERATISCKSSQSLLNSRTRENYLAWYQQKPGQPPKLLIYWASTRESGVPDRFSGSGSGTDFTLTISSLQAEDVAVYYCTQSYNLYTFGQGTKVEIKEVMYPPPYLDNERSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCEL

SEQ ID NO: 14 - protein, 21 aa, synthetic construct
  REGION 1..21: note = Ig kappa light chain leader
MDFQVQIFSFLLISASVIMSR

SEQ ID NO: 15 - protein, 22 aa, synthetic construct
  REGION 1..22: note = 2A self-cleaving peptide
GSGATNFSLLKQAGDVEENPGP

SEQ ID NO: 16 - protein, 676 aa, synthetic construct
  REGION 1..676: note = CAR-P2A-CCR (Ikappa-aCD19scFv-CD8a(hinge+tm)-CD3 (ic)-P2A-(CTLA-4 (ec+tm)-4-1BB(ic))
MDFQVQIFSFLLISASVIMSRDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMDFQVQIFSFLLISASVIMSRKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTKRGRKKLLYIFKQPFMRPVQTTQEEDGCSCRFPEEEEGGCEL

SEQ ID NO: 17 - protein, 21 aa, Homo sapiens
  REGION 1..21: note = transmembrane domain of human CTLA-4
FLLWILAAVSSGLFFYSFLLT

SEQ ID NO: 18 - protein, 189 aa, synthetic construct; identical to SEQ ID NO: 9
  REGION 1..189: note = CTLA-4(ec+tm)-4-1BB(ic)

SEQ ID NO: 19 - protein, 242 aa, synthetic construct
  REGION 1..242: note = aCD19(scFv), clone FMC63
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 20 - protein, 249 aa, synthetic construct
  REGION 1..249: note = aCD20(scFv), clone 2H7
MAQVKLQESGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGQGTTVTVSSGGGGSGGGGSGGGGSDIELTQSPTILSASPGEKVTMTCRASSSVNYMDWYQKKPGSSPKPWIYATSNLASGVPARFSGSGSGTSYSLTISRVEAEDAATYYCQQWSFNPPTFGGGTKLEIKRAAA

SEQ ID NO: 21 - protein, 246 aa, synthetic construct
  REGION 1..246: note = aCD22(scFv), clone m971
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 22 - protein, 181 aa, synthetic construct
  REGION 1..181: note = fused CD8 (ec+tm)-CD3 (ic)
TTTPAPRPPTPAPTIASQPLSLRPEACRPAAGGAVHTRGLDFACDIYIWAPLAGTCGVLLLSLVITLYCRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - protein, 423 aa, synthetic construct
  REGION 1..423: note = aCD19(scFv)-CD8 (ex+tm)-CD3 (ic)
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 24 - protein, 695 aa, synthetic construct
  REGION 1..695: note = aCD20(scFv)-aCD19(scFv)-CD8 (ex+tm)-CD3 (ic)
MAQVKLQESGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGQGTTVTVSSGGGGSGGGGSGGGGSDIELTQSPTILSASPGEKVTMTCRASSSVNYMDWYQKKPGSSPKPWIYATSNLASGVPARFSGSGSGTSYSLTISRVEAEDAATYYCQQWSFNPPTFGGGTKLEIKRAAAGGGGSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - protein, 112 aa, Homo sapiens
  source 1..112: note = intracellular domain of CD3 zeta
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2025/0375522 A1
(43) Pub. Date: Dec. 11, 2025

(54) CHIMERIC CHECKPOINT RECEPTOR FOR THE USE IN TREATMENT OF MALIGNANT B-CELL DISEASES

(71) Applicant: Universität zu Köln, Köln (DE)

(72) Inventor: Markus Chmielewski, Köln (DE)

(21) Appl. No.: 18/876,596

(22) PCT Filed: Jun. 28, 2023
(86) PCT No.: PCT/EP2023/067611

§ 371 Date: Dec. 18, 2024

(30) Foreign Application Priority Data
Jun. 29, 2022 (EP) 22181943.6

(51) Int. Cl. A61K 40/11; A61K 31/7088; A61K 35/17; A61K 40/42; A61P 35/00; C07K 14/00; C07K 16/28; C12N 5/0783; C12N 15/62; C12N 15/85

(52) U.S. Cl. CPC A61K 40/11; A61K 31/7088; A61K 35/17; A61K 40/421; A61K 40/4221; A61P 35/00; C07K 14/00; C07K 16/2827; C07K 16/2887; C12N 5/0636; C12N 15/62; C12N 15/85; A61K 2239/48; C07K 2317/31; C07K 2317/622; C07K 2319/03

(57) ABSTRACT

The invention is related to a chimeric checkpoint receptor (CCR) fusion protein, a nucleic acid molecule encoding said fusion protein, a vector comprising said nucleic acid molecule, a host cell comprising said nucleic acid molecule and/or expressing the fusion protein, a method for providing said host cell, a pharmaceutical composition comprising said fusion protein, nucleic acid molecule or host cell, and said products for use as a medicament and in the treatment of B cell lymphoma.

### TECHNICAL FIELD

The present invention relates to a chimeric checkpoint receptor (CCR) fusion protein, a nucleic acid molecule encoding said fusion protein, a vector comprising said nucleic acid molecule, a host cell comprising said nucleic acid molecule and/or expressing the fusion protein. a method for providing said host cell, a pharmaceutical composition comprising said fusion protein, nucleic acid molecule or host cell, and said products for use as a medicament and in the treatment of B cell lymphoma.

### BACKGROUND

The concept and advantages of CAR T cells are well known in the art and have become a focus of pharmaceutical research and development in recent years. A primary example of successful clinical application of CAR T cells in patients has been the regulatory approval of Tisagenlecleucel (Kymriah®) for the treatment of B-cell acute lymphoblastic leukemia in the US as well as in the EU. However, certain side effects and varying degrees of therapeutic success in patients may complicate the use of second generation CAR T cells such as Tisagenlecleucel in patients.

A major problem associated with the use of Tisagenlecleucel in patients is the generalized depletion of malignant as well as healthy CD19⁺ B lymphocytes, leading to a reported B cell aplasia and consequent hypogammaglobulinemia (Ali S et al., 2020 Oncologist, 25(2): e321-e327).

Furthermore, substantial neurotoxicity has been observed in patients treated with Tisagenlecleucel which may be attributed to the expression of CD19 on pericytes, thus causing an on-target off-tumor effect. The interaction between the CD19-specific CAR T cells and CD19⁺ pericytes has been reported to lead to an induction of an undesired release of IL-2 and IL-6 in such cells which may then lead to neurological symptoms (Parker KR et al., 2020 Cell, 183(1):126-142.e17.).

Both of IL-2 and IL-6 are considered alarm cytokines which may in turn recruit and activate other immune cells, such as macrophages, then contributing to increased IL-6 release after activation. In general, increased release of IL-6 can lead to cytokine release syndrome (CRS), which can trigger life-threatening situations in patients.

Increased IL-2 release has yet another negative impact on CAR T cell activity. IL-2 is critical for proliferation of regulatory T cells, which in turn can decrease T cell activity (Chinen T et al., 2016 Nat Immunol, 17(11): 1322-1333).

Maybe due to the above, the present inventor became aware that B-cell lymphoma (CD19⁺CD80⁺CD86⁺) bearing animals previously treated with Tisagenlecleucel CAR T cells developed tumor recurrence in about 50% of all cases. Re-treatment of those animals with tumor recurrence using Tisagenlecleucel CAR T cells did not result in therapeutic success.

Based on the aforementioned situation regarding the use of CAR T cells currently available, the present inventor thus set out to overcome the shortcomings of the prior art and provide and enable advantageous and improved CAR T cell therapies.

Thus, it is an object of the present invention to provide a CAR T cell therapy with increased safety and efficiency which avoids unspecific targeting of cells and cell types other than malignant B lymphocytes, such as healthy B lymphocytes and CD19⁺pericytes.

It is a further object of the present invention to provide a CAR T cell therapy which allows maintaining a population of healthy B lymphocytes and avoid neurotoxic symptoms which may appear upon using CAR T cell therapy as described in the prior art.

It is another object of the present invention to provide a CAR T cell therapy which reduces release of alarm cytokines such as IL-2 and IL-6 in order to avoid cytokine release syndrome as a prominent side effect.

It is also an object of the present invention to avoid tumor recurrence in B cell lymphoma patients and offer a promising therapeutic option for patients previously treated with conventional CAR T cell therapy and experiencing tumor recurrence.

### SUMMARY OF THE INVENTION

The aforementioned objects are solved by the aspects of the present invention as specified hereinafter.

According to the first aspect of the present invention, a fusion protein is provided comprising an extracellular domain comprising a polypeptide with specific affinity to CD80 and/or CD86, a transmembrane domain, and an intracellular domain comprising a co-stimulatory polypeptide.

In a preferred embodiment of the first aspect of the present invention, the polypeptide with specific affinity to CD80 and/or CD86 has an amino acid sequence which is at least 80% identical to the amino acid sequence of the extracellular domain of human CTLA-4 (SEQ ID NO: 1), or an amino acid sequence which is at least 80% identical to the amino acid sequence of the extracellular domain of human CD28 (SEQ ID NO: 2), or an amino acid sequence which is at least 80% identical to the amino acid sequence of the CD86-binding domain of the anti-CD86 antibody commonly referred to in the art as clone hu3D1 (SEQ ID NO: 5), preferably wherein the polypeptide with specific affinity to CD80 and/or CD86 has an amino acid sequence which is at least 80% identical to the amino acid sequence of the extracellular domain of human CTLA-4 (SEQ ID NO: 1).

In another preferred embodiment of the first aspect of the present invention, the transmembrane domain is suitable for insertion and anchoring of the fusion protein in the cell membrane of a mammalian cell, preferably wherein the transmembrane domain comprises the transmembrane region of one or more of the alpha, beta or zeta chain of the T-cell receptor, CTLA-4, CD28, CD3 epsilon, CD45, CD4, CD5, CD8 (e.g., CD8 alpha, CD8 beta), CD9, CD16, CD22, CD33, CD37, CD64, CD80, CD86, CD134, CD137, CD154, CD200, KIRDS2, 0X40, CD2, CD27, LFA-1 (CDlla, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7R .alpha., VLA1, ITGA1, CD49a, ITGA4, IA4, CD49D, ITGA6, VLA-6, CD49f. ITGAD, CDIId, ITGAE, CD103, ITGAL, CDIIa, LFA-1, ITGAM, CDIIb, ITGAX, CDIIc, ITGB1, CD29, ITGB2, CD18, LFA-1, ITGB7, TNFR2, DNAM1 (CD226), SFAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, LyI08), SEAM (SLAMF1, CD150, IPO-3), BLAME (SLAMF8), SELPLG (CD162), LTBR, and PAG/Cbp, or an amino acid sequence which is at least 80% identical thereto, preferably wherein the transmembrane domain comprises the transmembrane region of human CTLA-4 (SEQ ID NO: 1), or an amino acid sequence which is at least 80% identical thereto.

In one preferred embodiment of the first aspect of the present invention, the co-stimulatory polypeptide of the fusion protein comprises the intracellular domain of one or more of human 4-1BB (CD137; SEQ ID NO: 3), CD3ζ (SEQ ID NO: 25), CD28, ICOS, OX-40, BTLA, CD27, CD30, GITR, Myd88-CD40, KIR2DS2 and HVEM, or an amino acid sequence which is at least 80% identical thereto, preferably wherein the co-stimulatory polypeptide of the fusion protein comprises the intracellular domain of human 4-1BB (CD137: SEQ ID NO: 3), or an amino acid sequence which is at least 80% identical thereto, or preferably wherein the co-stimulatory polypeptide comprises the intracellular domain of CD3ζ (SEQ ID NO: 25), or an amino acid sequence which is at least 80% identical thereto.

In another preferred embodiment of the first aspect of the present invention, the polypeptide with specific affinity to CD80 and/or CD86 has an amino acid sequence comprising the extracellular domain of human CTLA-4 (SEQ ID NO: 1), or an amino acid sequence which is at least 80% identical thereto, and the intracellular domain comprises a co-stimulatory peptide comprising the intracellular domain of human 4-1BB (CD137; SEQ ID NO: 3), or an amino acid sequence which is at least 80% identical thereto.

According to the second aspect of the present invention, a nucleic acid molecule is provided encoding the fusion protein of the first aspect of the present invention.

According to the third aspect of the present invention, a vector is provided comprising the nucleic acid molecule of the second aspect of the present invention.

According to the fourth aspect of the present invention, a host cell is provided comprising the nucleic acid molecule of the second aspect of the present invention or the vector of the third aspect of the present invention.

In a preferred embodiment of the fourth aspect of the present invention, the host cell is transduced with the nucleic acid molecule of the second aspect of the present invention or the vector of the third aspect of the present invention, or wherein the nucleic acid molecule of the second aspect of the present invention or the vector of the third aspect of the present invention is stably integrated into the genome of the host cell.

According to the fourth aspect of the present invention, a host cell is also provided stably or transiently expressing a fusion protein according to the first aspect of the present invention.

In one specific preferred embodiment of the fourth aspect of the present invention, the host cell stably or transiently expresses a (co-expressed) protein comprising an extracellular domain with specific affinity to CD19 and an intracellular co-stimulatory domain, preferably wherein the extracellular domain with specific affinity to CD19 comprises at least an antigen-binding fragment of an anti-CD19 antibody, preferably wherein the extracellular domain with specific affinity to CD19 comprises an anti-CD19 scFv.

In one embodiment of the specific preferred embodiment of the fourth aspect of the present invention, the intracellular costimulatory domain of the co-expressed (CAR) protein comprises an amino acid sequence of the intracellular domain of CD3ζ (SEQ ID NO: 25), or an amino acid sequence which is at least 80% identical thereto. In a preferred embodiment of the fourth aspect of the present invention, the host cell is a human CD8⁺ T cell. According to the fifth aspect of the present invention, a method of providing a host cell of the fourth aspect of the present invention is provided comprising transducing a host cell with a nucleic acid molecule of the second aspect of the present invention or the vector of the third aspect of the present invention, cultivating the transduced host cell of the previous step in a suitable medium allowing growth of the cell and expression of the fusion protein encoded by said nucleic acid molecule or said vector, and collecting the host cells from the medium.

According to the fourth aspect of the present invention, a host cell is also provided which is obtainable by the method of the fifth aspect of the present invention.

According to the sixth aspect of the present invention, a pharmaceutical composition is provided comprising a fusion protein of the first aspect of the present invention, a nucleic acid molecule of the second aspect of the present invention, a vector of the third aspect of the present invention. and/or a host cell of the fourth aspect of the present invention.

According to the seventh aspect of the present invention, a fusion protein of the first aspect of the present invention, a nucleic acid molecule of the second aspect of the present invention, a vector of the third aspect of the present invention, a host cell of the fourth aspect of the present invention of a pharmaceutical composition of the sixth aspect of the present invention is provided for use as a medicament.

According to the eighth aspect of the present invention, a fusion protein of the first aspect of the present invention, a nucleic acid molecule of the second aspect of the present invention, a vector of the third aspect of the present invention, a host cell of the fourth aspect of the present invention or a pharmaceutical composition of the sixth aspect of the present invention is provided for use in the treatment of B cell lymphoma.

In a preferred embodiment of the eighth aspect of the present invention, the B cell lymphoma is a non-Hodgkin lymphoma selected from the group comprising marginal zone B cell lymphoma (MZL), mucosa-associated lymphatic tissue lymphoma (MALT), small lymphocytic lymphoma/chronic lymphocytic leukemia (SLL/CLL), mantle cell lymphoma (MCL), Burkitt's lymphoma, lymphoplasmacytic lymphoma, Waldenström's macroglobulinemia, nodal marginal zone B cell lymphoma (NMZL), splenic marginal zone lymphoma (SMZL), and diffuse large B cell lymphoma (DLBCL), preferably the B cell lymphoma is diffuse large B cell lymphoma (DLBCL).

According to the ninth aspect of the present invention, a kit or kit-in-parts is provided comprising a fusion protein of the first aspect of the present invention, a nucleic acid molecule of the second aspect of the present invention, a vector of the third aspect of the present invention, or a host cell of the fourth aspect of the present invention.

### DESCRIPTION OF FIGURES

FIG. 1 shows (A) a schematic diagram depicting the modular composition of the CAR/CCR expression cassette of the present invention (Lk, light chain kappa signal sequence; anti-CD19scFv, CD19-specific single chain (heavy and light) fragment variable: CD8a hinge, CD8 alpha hinge domain; CD8a tm, CD8 alpha transmembrane domain; CD3ζ (ic), CD3 zeta intracellular signaling domain; P2A, 2A peptide; CTLA-4 ex, Cytotoxic T-Lymphocyte Associated protein extracellular domain; CTLA-4 tm, Cytotoxic T-Lymphocyte Associated protein transmembrane domain; 4-1BB (ic), 4-1BB (CD137) intracellular signaling domain; (B) a schematic diagram depicting the expression and localization of the CAR/CCR construct on the surface of a T cell; (C) the expression of an anti-CD19(FMC63)-CD8a(ex)-CD3ζ(tm+ic)-CAR and a CTLA4(ex)-4-1BB(tm+ic)-CCR on retrovirally engineered human CD3⁺ T cells; CAR/CCR expression was monitored by flow cytometry using a phycoerythrin-labeled anti-idiotypic antibody and a PE-Cy-7-conjugated CTLA-4-specific antibody; mock-transduced human CD3³⁰ T cells served as controls (gray area).

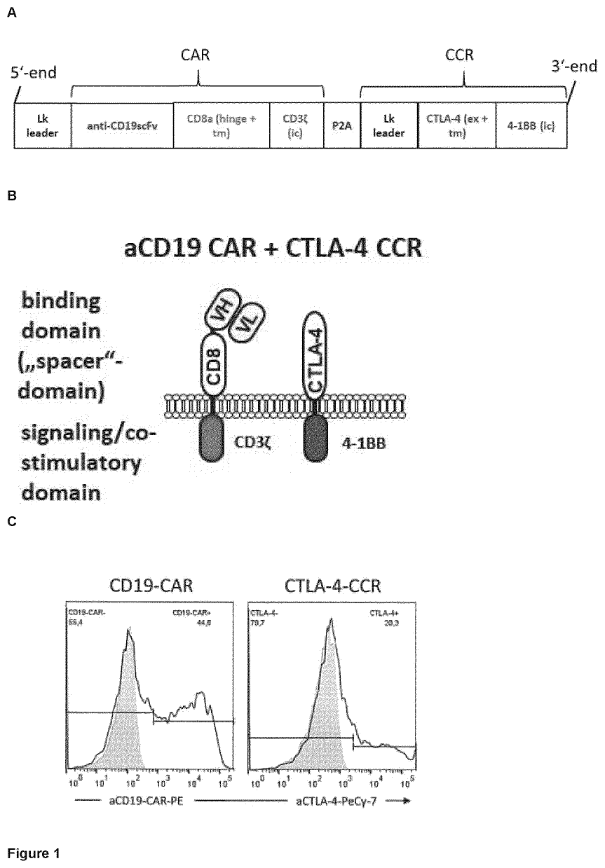

FIG. 2 shows high expression levels of both checkpoint-receptor ligands CD80 and CD86 on the surface of relapsed B-cell lymphoma cells after CD19-CAR-T cell therapy: tumor biopsies from patients suffering from DLBCL were collected before and upon CAR T cell treatment (Kymriah, Novartis) and fixed in 10% Neutral Buffered Formalin (NBF), processed and embedded in paraffin; formalin-fixed, paraffin-embedded (FFPE) tissues; fixed tissue sections were stained for CD80 and CD86 expression using HRP-conjugated CD80 and CD86 antibodies; after the detection step, colored chromogen DAB was added, and the slides were counterstained with Hematoxylin; recurrent DLBCL cells after cellular CAR T cell immunotherapy revealed increased expression of the immune checkpoint ligands CD80 and/or CD86 compared to CAR T naïve samples derived from the same patient.

FIG. 3 shows the analysis of primary DLBCL and CLL tumor cells for the expression of both CD80 and CD86; primary DLBCL cells exhibit increased expression of CD80 and/or CD86 in contrast to primary CLL cells and healthy B cells (controls).

FIG. 4 shows reduced IL-2 (A) and IL-6 (B) release by human CD19-specific CAR/CCR T cells of the present invention upon co-cultivation with human CD19⁺ B cells in presence of CD11b⁺ monocytes; primary T cells were engineered to express CD19-CAR/CTLA-4-CCR constructs (first bar from the left) and co-cultivated with CD19⁺ B cells for 24 h (in a ratio of 100 CAR T cells: 100 B cells: 1 monocyte): IL-2 released by CAR T cells and IL-6 released by monocytes into the supernatant were detected by ELISA; T cells engrafted with a CD19-specific CAR of the second generation (α-CD19-CD28-CD3ζ; second bar from the left), of the first generation (α-CD19-CD3ζ; central bar), and untransduced cells (second bar from the right) were used as controls; further shown is (C) a reduced cytotoxic effect on primary human B cells and (D) a reduced IFN-γ release by human CD19-specific CAR/CCR T cells of the present invention upon co-cultivation with primary human CD19⁺B cells: primary T cells were engineered to express CD19-CAR/CTLA-4-CCR constructs (first bar from the left) and co-cultivated with primary CD19⁺ B cells for 18 h in a effector-target ratio of 1:10 (E:T) T cells genetically modified to express 1^{st} and 2^{nd} generation CAR contructs and non-modified T cells (Mock) were used as controls.

FIG. 5 shows reduced IFN-γ release by human CD19-specific CAR/CCR T cells of the present invention upon co-cultivation with human CD19 and CD248 expressing pericytes (A); therefore, primary human vascular wall-typical mesenchymal stem cells (VW-MSC) were differentiated into CD19 and CD248 expressing pericyles by culture in media supplemented with TGF-β3 for 2 weeks; finally, differentiated CD19+CD248+pericytes were isolated by BD FACSAria III sorter. (B) Next, T cells were equipped with CAR (2^{nd} gen) or CAR/CCR constructs and co-cultivated with CD19⁺CD248⁺ pericytes (in a ratio of 1 CAR T cell: 1 pericyte) for 24 h: finally, superatants from this experiment were collected and analysed for IFN-y by ELISA; superatants from mock-transduced T cells and (CAR) T cells cultivated without (w/o) pericytes were used as controls.

FIG. 6 shows that CD19-CAR/CTLA-4-CCR T cells efficiently eliminate CD80⁺ CD86⁺ cells of the aggressive B cell lymphoma cell line Raji; primary T cells were engineered to express CD19-CAR/CTLA-4-CCR constructs (left bar in each panel) and co-cultivated with CD19⁺ CD80⁺ CD86⁺ cells of the cell line Raji in a ratio of 1:4 (effector to target cells) for 24 to 120 hours; cytotoxic effects on tumor cells were analyzed using a HIDEX ELISA reader; T cells engrafted with a CD19-specific CAR of the second generation (α-CD19-CD28-CD3ζ; central bar in each panel) and of the first generation (α-CD19-CD3ζ; right bar in each panel) were used as controls.

FIG. 7 shows that CD19-CAR/CTLA-4-CCR T cells efficiently eliminate B cell lymphoma cells and enhance tumor-free survival; CD19⁺ CD80⁺ CD86⁺ cells of the cell line Raji were intravenously injected into RAG2^{-/-}0 common γ^{-/-} mice and treated on day 5 with CD19-CAR/CTLA-4-CCR T cells; T cells engineered to express second generation CD19-CAR (Kymriah, Novartis), mock transduced T cells were used as controls.

FIG. 8 shows IFN-y release by CD19CAR/CTLA-4CCR, CD19CAR (2nd gen) or CD19CAR (1st gen) T cells after co-culture with CD19-expressing primary DLBCL cells (CD80^{high}CD86^{high}), DLBCL cell line SU-DHL-10 (CD80^{high}CD86^{high}), DLBCL cell line DOHH-2 (CD80^{high}CD86^{low}), DLBCL cell line Oci-Ly1 (CD80^{low}CD86^{high}) or DLBCL cell line Oci-Ly19 (CD80^{low}CD86^{low}); cells were co-cultivated with CAR/CCR, CAR (1st gen) or CAR (2^{nd} gen) T cells for 48 hours at 37° C. Target cells co-cultivated with mock transduced T cells (Mock) or without effector T cells (No T cells) served as control (ns=not significant, *p<0.05, ** p<0.005).

FIG. 9 shows IL-2 release by CD19CAR/CTLA-4CCR, CD19CAR (2nd gen) or CD19CAR (1st gen) T cells after co-culture for 48 hours at 37° C. with CD19 expressing primary DLBCL cells (CD80^{high}CD86^{high}), DLBCL cell line SU-DHL-10 (CD80^{high}CD86^{high}), DLBCL cell line DOHH-2 (CD80^{high}CD86^{high}), DLBCL cell line Oci-Ly1 (CD80^{low}CD86^{high}) or DLBCL cell line Oci-Ly19 (CD80^{low}CD86^{low}). Target cells co-cultivated with mock transduced T cells (Mock) or without effector T cells (No T cells) served as control (ns=not significant, *p<0.05. ** p<0.005).

FIG. 10 shows a luminescence plot of xenograft mouse studies, based on Raji-based lymphoma bearing Rag2−/− IL-2rg −/− mice, treated with 8.0×10⁶ CAR/CCR, CAR (2nd gen) or mock T cells and evaluated for tumor growth using bioluminescence; Raji expressing CD19/CD80/CD86 were genetically modified to express firefly Luciferase and intravenously injected into immunocompromised mice; three days upon tumor transplantation, mice were divided into three groups and treated with effector T cells (2nd gen CAR or CAR/CCR); a group of mice treated with mock-transduced T cells were used as control; tumor progression was monitored by using IVIS bioluminescence device; mice were sacrificed at tumor burden cutoff or when showing paralysis.

FIG. 11 shows IL-6 release by activated macrophages in vitro; CAR T cells were cultivated in the presence of CD19/80/86 expressing tumor cell line SU-DHL10 and separated human CD11b* macrophages of the same healthy donor; 1×10⁵ CAR/CCR, CAR (2nd gen) or non-transduced (nt) T cells (+T) were cultivated in the presence of 3.25×10⁴ macrophages (+M) at 37° C. for 24 hours; finally, supernatant were harvested and investigated for IL-6 release by ELISA; tumor cells cultivated in the presence/absence of macrophages and/or (CAR/CCR, CAR, nt) T cells served as controls.

FIG. 12 schematically shows the design of CAR and CAR/CCR constructs used herein; (A) a construct according to the present invention with a CD20-specific CAR (comprising an anti-CD20 scFv as the extracellular domain and CD3ζ as the intracellular signaling domain comprising a co-stimulatory polypeptide), and a CCR comprising CTLA-4 as the extracellular domain comprising a polypeptide with specific affinity to CD80 and/or CD86, and the intracellular domain of 4-1BB as the intracellular domain comprising a co-stimulatory polypeptide; (B) a construct according to the present invention with a CD19-specific CAR (comprising an anti-CD19 scFv as the extracellular domain and CD34 as the intracellular signaling domain comprising a co-stimulatory polypeptide), and a CCR comprising an anti-CD86 scFv as the extracellular domain comprising a polypeptide with specific affinity to CD86, and the intracellular domain of 4-1BB as the intracellular domain comprising a co-stimulatory polypeptide: (C) a CD20-specific CAR of the second generation as known in the art; and (D) a CD 19-specific CAR of the second generation as known in the art.

FIG. 13 shows an analysis of the cytotoxic effects (A;C) and of the release of proinflammatory cytokine IFN-y (B;D) by T cells modified to express the constructs shown in FIG. 12 in the DLBCL (diffuce large B-cell lymphoma) cell line SUDHL-10 (A;B) and in the MCL (mantle cell lymphoma) cell line JeKo-1R (C; D).

FIG. 14 shows an analysis of the cytotoxic effects (A) and of the release of proinflammatory cytokine IFN-γ (B) by T cells modified to express the constructs shown in FIG. 12 in a JeKo-1R cell line with 99.9% reduced/diminished CD19 expression (verified by RT-PCR; data not shown).

FIG. 15 schematically shows the design of a reverse switch, wherein the intracellular signaling domains comprising the co-stimulatory polypeptide have been exchanged between CAR and CCR; (A) a construct with a CD19-specific CAR (comprising an anti-CD19 scFv as the extracellular domain and CD3ζ as the intracellular signaling domain comprising a co-stimulatory polypeptide) in combination with a CCR comprising CTLA-4 as the extracellular domain comprising a polypeptide with specific affinity to CD80 and/or CD86, and the intracellular domain of 4-1BB as the intracellular domain comprising a co-stimulatory polypeptide, and (B) a reverse switch leading to a CAR and a CCR as in (A) with the difference that the CAR comprises the intracellular domain of 4-1BB as the intracellular domain comprising a co-stimulatory polypeptide, and the CCR comprises CD3ζ as the intracellular signaling domain comprising a co-stimulatory polypeptide; (C) demonstrates in flow cytometry histograms that human T cells expressing the molecules described in (A) and (B) both led to efficient cytotoxicity on a Su-DHL-5 B cell lymphoma cell line; (D) showing the FACS gating strategy.

FIG. 16 schematically shows the design of (A) a fully murine CAR/CCR construct according to the present invention comprising a CD19-specific CAR (comprising an anti-CD19 scFv as the extracellular domain and CD3ζ as the intracellular signaling domain comprising a co-stimulatory polypeptide), and a CCR comprising CTLA-4 as the extracellular domain comprising a polypeptide with specific affinity to CD80 and/or CD86 and the intracellular domain of 4-1BB as the intracellular domain comprising a co-stimulatory polypeptide; (B) a fully murine CD19-specific CAR of the second generation as known in the art; and (C) results on cell count and IFN-α release observed when bringing T cells expressing the constructs shown in (A) and (B) in contact in vitro with murine CD19⁺ CD80⁺ CD86⁺ B cell lymphoma cells, or with primary CD19⁺ CD80⁻ CD86⁻ B cells.

FIG. 17 shows in vivo effects of T cells expressing the constructs described in FIG. 16 above in mice of the C57bl/6 (wt) mouse strain with regard to the naïve B cell population over time in peripheral blood and the spleen (rightmost graph) shown as (A) ratios, and (B) FACS histograms (top: CAR of the second generation as in FIG. 16(B): bottom: CAR/CCR construct as in FIG. 16(A)), (C) FACS gating strategy.

### DETAILED DESCRIPTION OF THE INVENTION

The present invention is based on the recognition that a discrimination between healthy and malignant B cells as well as between other cell types such as pericytes and malignant B cells is made possible by employing a co-stimulatory chimeric checkpoint receptor (CCR) which recognizes checkpoint ligands on malignant cells such as CD80 and CD86.

The present inventor found that the neurotoxicity observed during therapeutic treatment of patients with Kymriah may be due to the fact that CD19 is also expressed on neuronal pericytes (cf. Parker KR et al., 2020; supra). Undesired toxicity of CAR T cells directed against CD19 has previously been addressed by other researchers, in some cases also employing a co-stimulatory CCR (cf. Liao Q et al., 2020 Biomarker Research, 8:57 and Blaeschke F et al., 2021 Blood Cancer Journal, 11:108).

However, both of Liao et al., 2020 and Blaeschke et al., 2021 have used a CCR directed against PD-L1 (α-PD-L1-scFv in case of Liao et al., and PD-1 in case of Blaeschke et al.) in combination with a CD19-CAR. This combination appears unsuitable to abolish neurotoxicity, since it was already previously reported elsewhere that PD-L1 is also expressed on the surface of neuronal pericytes (Domev et al., 2014 Stem Cells Transl Med., 3(10):1169-1181) and may provide a co-stimulatory signal to the aforementioned CCR fusion proteins.

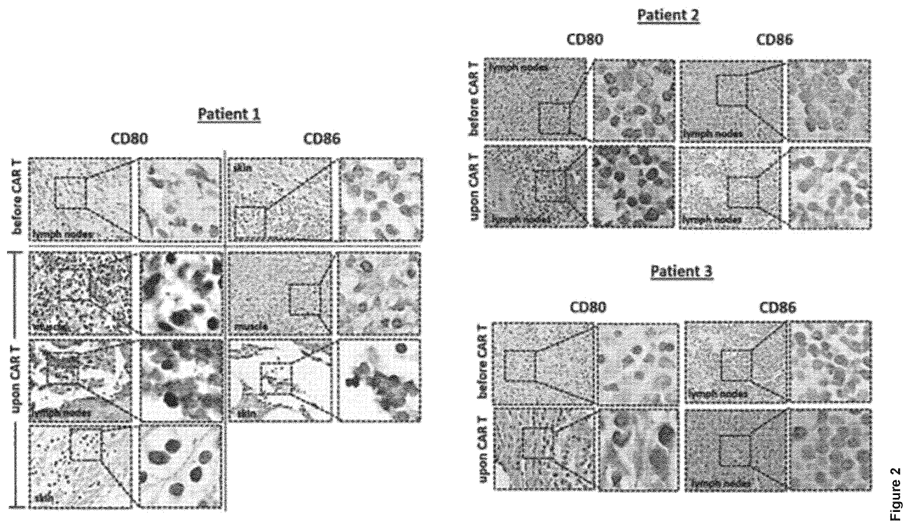

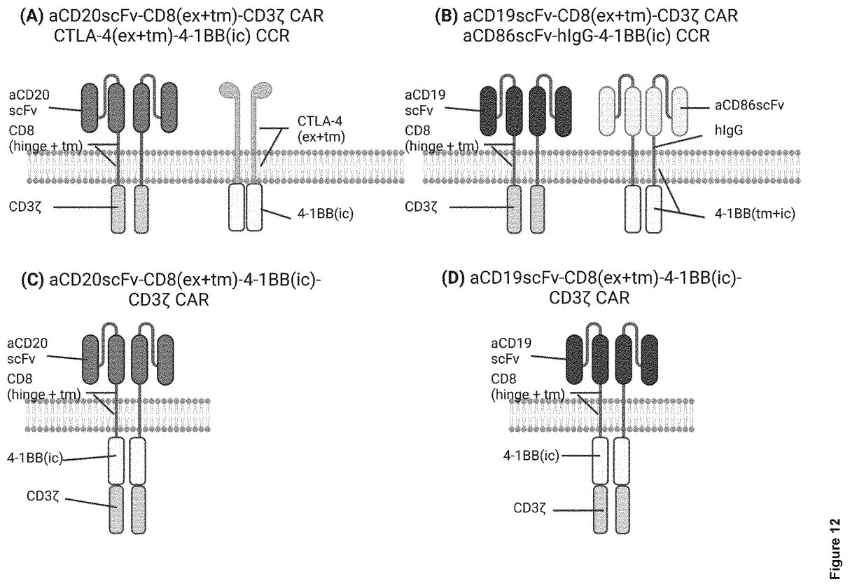

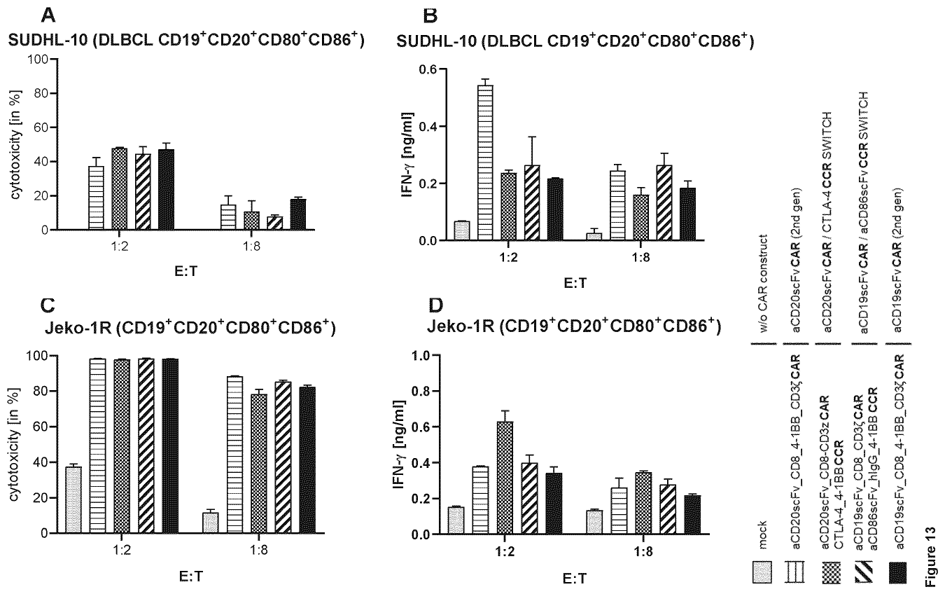

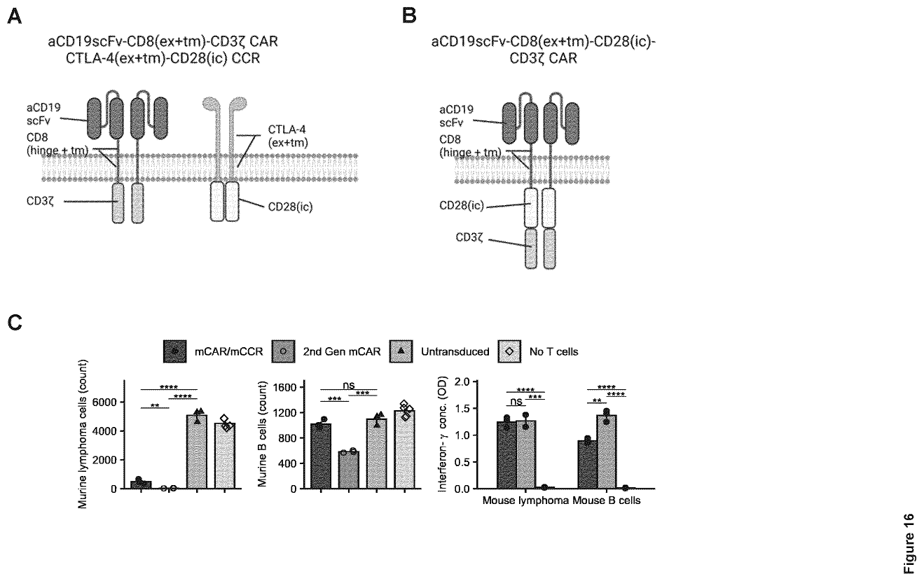

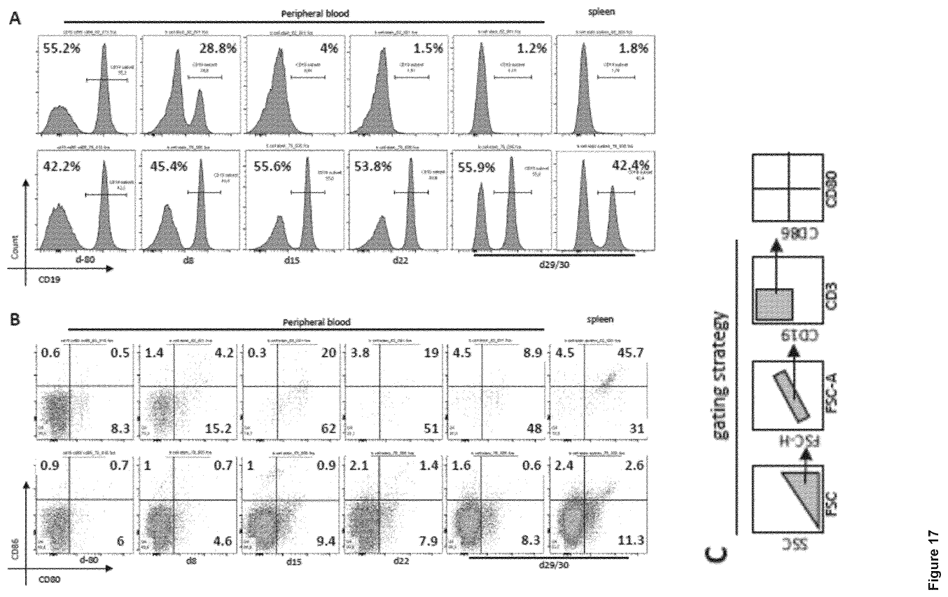

One particular advantage of using the CCR of the present invention directed against CD80 and/or CD86 aside from a reduced likelihood of neurotoxicity is that B-cell lymphoma cells which have previously been treated with CD19-specific CAR T cells show increased expression of CD80 and/or CD86 (see FIG. 2). Accordingly, any moiety targeting CD80 and/or CD86 may suitably be used as part of the CCR according to the present invention (see FIGS. 12 and 13), and may serve to reduce any undesired targeting of primary B cells in vitro (see FIG. 16) and in immunocompetent mice of the C57bl/6N (wt) mouse strain (see FIG. 17).

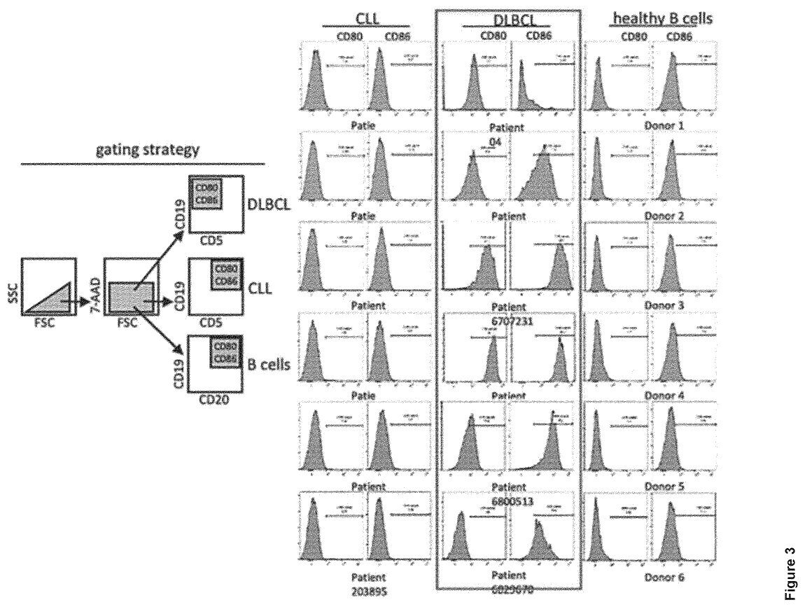

A similar observation has been made by the present inventors for primary B cell lymphoma cells (i.e. primary diffuse large B-cell lymphoma (DLBCL) cells; see FIG. 13(A) and (B)) in contrast to leukemia cells (i.e. chronic lymphocytic leukemia (CLL) cells; see FIG. 3). This suggests that even primary B cell lymphoma cells are particularly accessible for CAR-T cells of the present invention which profit from the co-stimulus by CD80 and/or CD86.

Thus, the present invention for the first time enables promising treatment options for patients suffering from B cell lymphoma in general, and also from tumor recurrence after initial (and unsuccessful) therapy with Kymriah or the like. The strategy of the present invention is based on expressing a recombinant CTLA-4 co-receptor (CCR) in addition to the CAR, wherein the CCR binds and neutralizes the checkpoint ligands CD80 and CD86 on B-cell tumor cells.

By splitting the signaling moiety to both receptors CD19-CAR-CD3ζ and CTLA-4-CCR-4-1BB, CAR-T cells are fully activated only when CD80 and/or CD86 is expressed together with CD19 on the same target cell. This avoids unspecific and off-tumor activation of the T cells. Based on the experimental results provided herein, these advantageous effects are not limited to the specific construct mentioned above but can also be observed with different modules as long as they fulfill the required binding specificity and/or the signaling capability.

In this regard, a CD19-binding extracellular domain of the co-expressed (CAR) protein may be functionally replaced by a different CD19-binding moiety, a CD20-binding moiety, a CD22-binding moiety, or yet another moiety binding to cell surface molecule found on cancer cells or extracellular cancer biomarker. Along the same lines, the extracellular domain of CTLA-4 may also be functionally replaced by a CD80- and/or CD86-binding moiety, such as CD28 or any CD80/CD86-binding moiety derived from an anti-CD80 or anti-CD86 antibody.

These recognitions which are based on the experimental data disclosed herein illustrate the recognition that binding to a tumor biomarker such as CD19 in combination with binding to CD80 or CD86 will credibly lead to the benefits obtained with the present invention.

Furthermore, beyond the effects caused by the specific examples of the present invention, it will be evident to the skilled person that the intracellular stimulatory and co-stimulatory domains may be replaced by functional equivalents as disclosed herein as long as they are able to provide the intracellular signal required for T cell activation. Accordingly, it is not only possible to switch the (co-) stimulatory moieties between the fusion protein and the co-expressed (CAR) protein but any (co-) stimulatory domains or combinations thereof which are known to convey the required intracellular signaling may be used within the present invention.

However, in order to provide the required intracellular signaling within the CAR T cells necessary to cause T cell activation and killing of tumor cells, at least one intracellular domain of the fusion protein and/or of the co-expressed (CAR) protein must contain one or more immunoreceptor tyrosine-based activation motifs (ITAMs; cf. Zhang et al., 2017 Biomarker Research, 5:22). While the two most prominent examples and most commonly used ITAM-containing domains (FcεRlγ (ic) with one ITAM. and CD3ζ (ic) with three ITAMs) may preferably be used in the context of the present invention, domains with synthetic and artificially designed ITAMs which perform an analogous function may also be suitably used and are encompassed by the present invention.

In embodiments, wherein an ITAM-containing domain is provided as part of the fusion protein, a co-stimulatory domain as described herein is provided as part of the co-expressed (CAR) protein. In embodiments, wherein an ITAM-containing domain is provided as part of the co-expressed (CAR) protein, a co-stimulatory domain as described herein is provided as part of the fusion protein. Also, it had previously been reported that IL-6 may be released by antigen-presenting cells (APC), such as macrophages or pericytes, which may then cause an off-tumor cytokine release syndrome (CRS) in patients. The PD1-CD28-fusion CCR construct which has been used in Liao et al., 2020 as well as in Blaeschke et al., 2021 has recently been reported to lead to such an (undesired) increased release of IL-2, IFN-γ and/or TNF.

These cytokines may serve to recruit and activate other immune cells, such as macrophages, which then contribute to increased release of IL-6 and the onset of CRS as well as MAS-L (cf. Kennedy VE et al., Blood (2020) 136 (Supplement 1): 7-8.). This is particularly pronounced when using CD28 in combination with the CD3ζ intracellular domain. Also, an increased release of IL-2 induces proliferation of regulatory T cells which may reduce T cell activity of CAR T cells (cf. for example Chinen et al., Nat Immunol, 2016; 17(11): 1322-1333). This may occur pronouncedly when employing the strategies proposed by Liao et al, 2020 and Blaeschke et al., 2021.

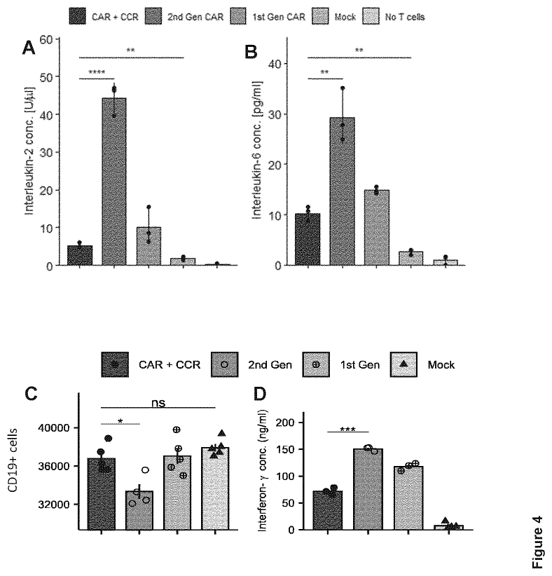

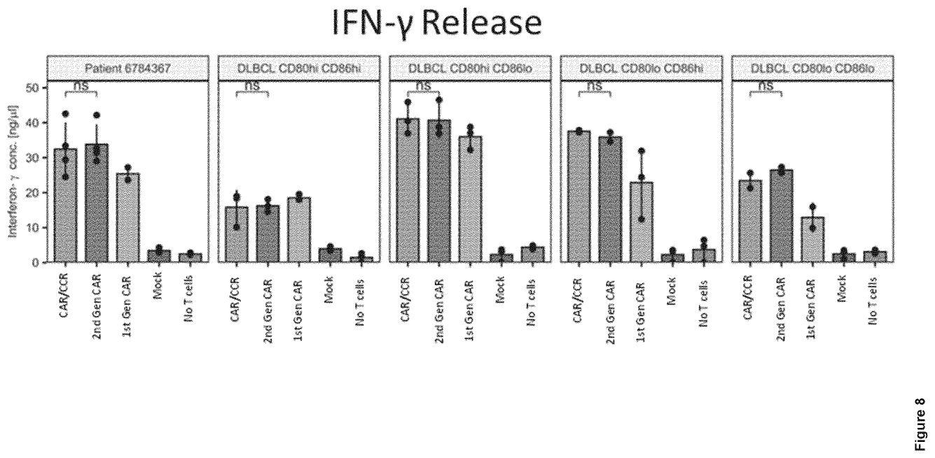

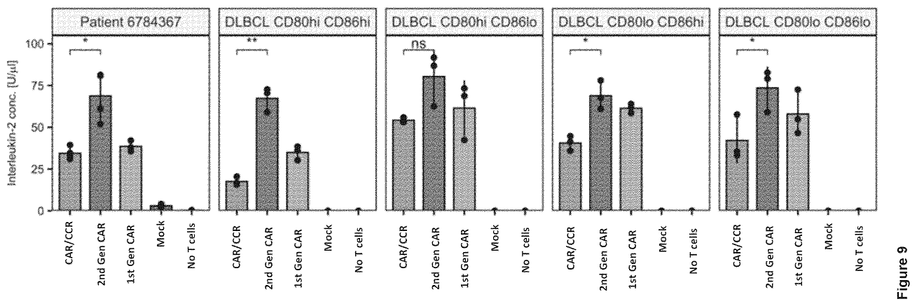

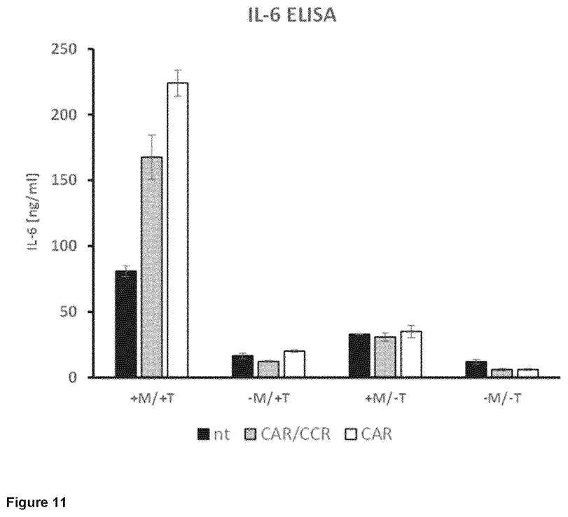

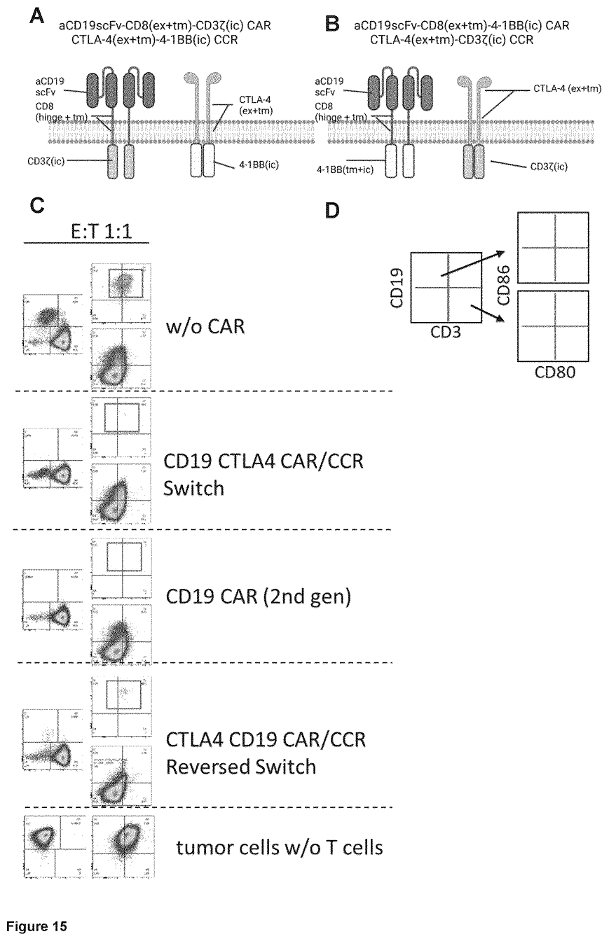

Both of the aforementioned problems appear to be less pronounced when using the strategy of the present invention which involves the use of an alternative intracellular signaling domain such as 4-1BB (see FIGS. 4, 5, 8, 9 and 11 herein) or CD3ζ (see FIG. 15).

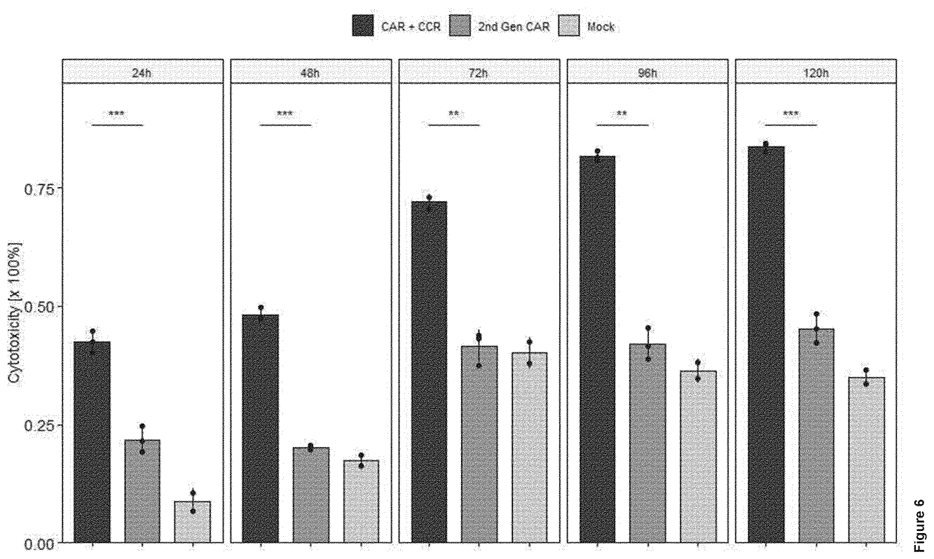

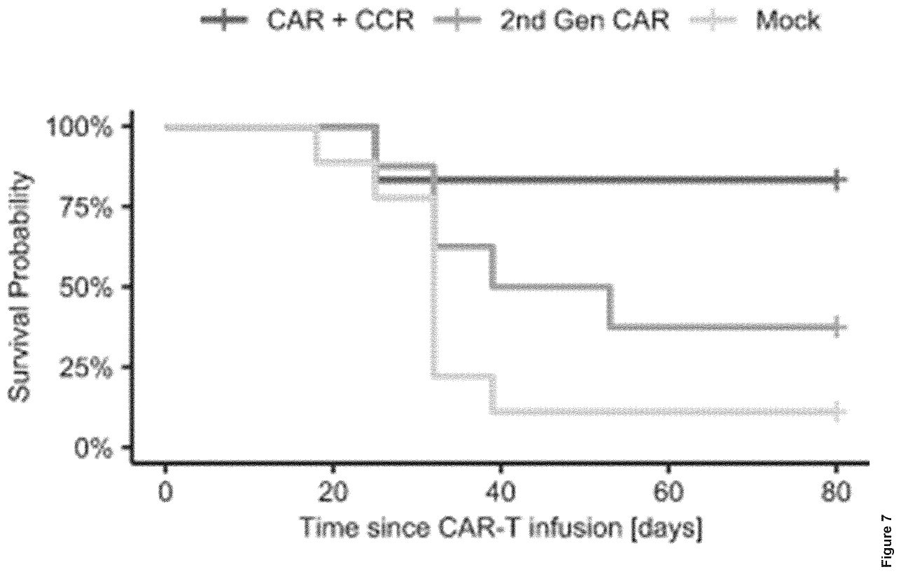

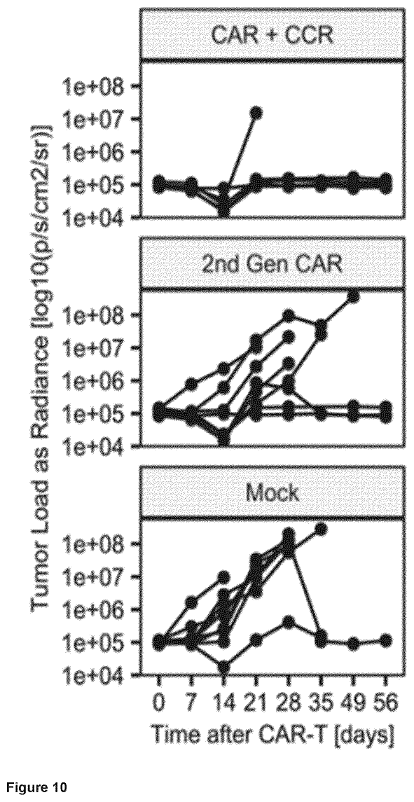

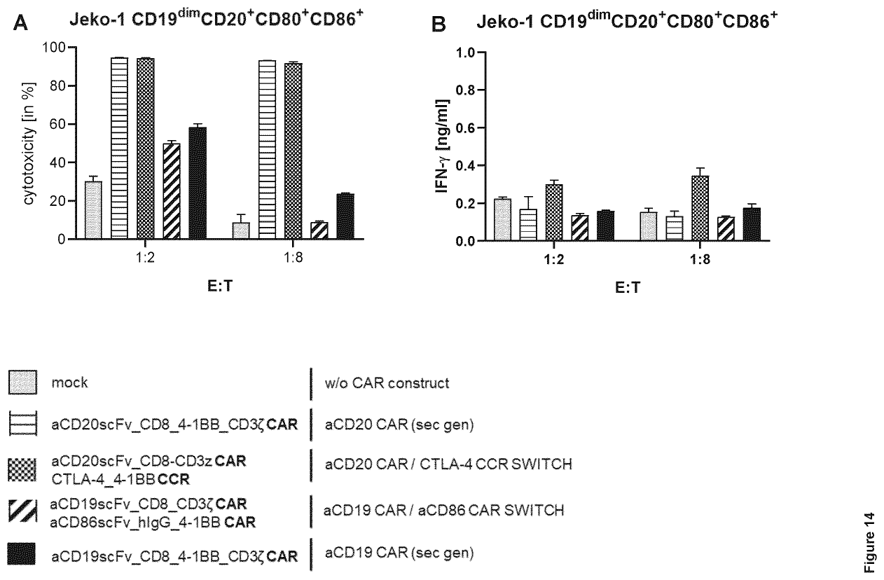

The strategy of the present invention leads to efficient elimination of CD19⁺, CD80⁺ and/or CD86⁺ B-cell lymphomas, even upon recurrence after initial CAR T cell treatment (see FIG. 6), e.g. by addressing CD20 as a tumor biomarker (see FIG. 14). This efficient elimination of tumor cells was further demonstrated to lead to an increased length of tumor-free survival (see FIG. 7), and reduced tumor progression in a xenograft mouse model (see FIG. 10).

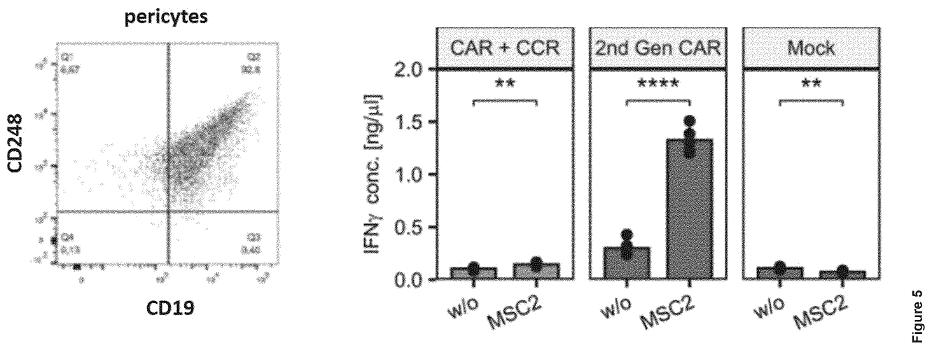

Various side effects can be limited or overcome by using the strategy of the present invention. IL-2 and IL-6 release is reduced (see FIGS. 4, 5. 9 and 11), off-tumor and neurotoxic effects prevented and a pool of healthy B lymphocytes may persist after CAR T cell therapy (as demonstrated in vitro by the results of FIG. 4(D) and FIG. 16(C) as well as in vivo by the results of FIG. 17).

Thus, the present invention provides a CAR T cell therapy with increased safety and efficacy, avoiding unspecific targeting of cells other than malignant B lymphocytes, maintains a population of healthy B lymphocytes in treated patients, largely avoids neurotoxic symptoms such as those described as immune effector cell-associated neurotoxicity syndrome (ICAN) which are commonly observed with therapies of the prior art, reduces release of alarm cytokines and development of cytokine release syndrome, and is able to avoid and specifically target tumor recurrence in B cell lymphoma patients.

Accordingly, in a first aspect of the invention, a fusion protein is provided comprising an extracellular domain comprising a polypeptide with specific affinity to CD80 and/or CD86, a transmembrane domain, and an intracellular domain comprising a co-stimulatory polypeptide.

As an extracellular domain of the fusion protein of the present invention comprising a polypeptide with specific affinity to CD80 and/or CD86, the extracellular domain of CTLA-4 (CD152) (UniProtKB-P16410) is preferred. Alternatively, the extracellular domain of CD28 (UniProtKB-P10747) may preferably be used due to its specific affinity to CD80 and/or CD86.

Alternatively, antibodies or antibody fragments with specific affinity to CD80 and/or CD86 may be preferred. In particular, an amino acid sequence which is at least 80% identical to the amino acid sequence of the CD86-binding domain of the anti-CD86 antibody commonly referred to in the art as cione hu3D1 (SEQ ID NO: 5) may be used as the polypeptide with specific affinity to CD86.

In one embodiment, an antibody or antibody fragment with specific affinity to CD80 is used as part of the extracellular domain of the fusion protein of the present invention, preferably derived from the CD80-binding domain of the anti-CD80 antibody commonly referred to in the art as clone hu1F1, more preferably of the amino acid sequence:

(SEQ ID NO: 4)
QLVQSGAEVKKPGASVKVSCKPSGFNIKDYYMHWVRQAPGQGLEWI
GWIDPENGNTLYDPKFQGKATITADTSTSTAYMELSSLRSEDTAV
YYCAREGLFFAYWGQGTLVTVSSGGGGSGGGGSGGGGSDIQMTQS
PSSLSASVGDRVTITCSVSSSISSSNLHWYQQKPGKAPKPLIYGT
SNLASGVPSRFSGSGSGTDYTLTISSLQPEDVATYYCQQWSSYPL
TFGQGTKVEIK.

In another embodiment, an antibody or antibody fragment with specific affinity to CD86 is used as part of the extracellular domain of the fusion protein of the present invention, preferably derived from the CD86-binding domain of the anti-CD86 antibody commonly referred to in the art as cione hu3D1, more preferably of the amino acid sequence:

(SEQ ID NO: 5)
QVQLVQSGAEVKKPGSSVKVSCKASGYTFTDYAIQWVRQAPGQGL
EWIGVINIYYDNTNYNQKFKGKATMTVDKSTSTAYMELSSLRSED
TAVYYCARAAWYMDYWGQGTLVTVSSGGGGGGGGSGGGGSDIVLT
QSPDSLAVSLGERATISCKSSQSLLNSRTRENYLAWYQQKPGQPP
KLLIYWASTRESGVPDRFSGSGSGTDFTLTISSLQAEDVAVYYCT
QSYNLYTFGQGTKVEIK.

In one preferred embodiment, a combination of an antibody or antibody fragment directed against CD80 with an antibody or antibody fragment directed against CD86 may be used as part of the extracellular domain of the fusion protein of the present invention, preferably linked by a conventional linker sequence, more preferably having the amino acid sequence:

(SEQ ID NO: 6)
QLVQSGAEVKKPGASVKVSCKPSGFNIKDYYMHWVRQAPGQGLEW
IGWIDPENGNTLYDPKFQGKATITADTSTSTAYMELSSLRSEDTA
VYYCAREGLFFAYWGQGTLVTVSSGGGGSGGGGSGGGGSDIQMTQ
SPSSLSASVGDRVTITCSVSSSISSSNLHWYQQKPGKAPKPLIYG
TSNLASGVPSRFSGSGSGTDYTLTISSLQPEDVATYYCQQWSSYP
LTFGQGTKVEIKGGGGSGGGGSGGGGSGGGGSQVQLVQSGAEVKK
PGSSVKVSCKASGYTFTDYAIQWVRQAPGQGLEWIGVINIYYDNT
NYNQKFKGKATMTVDKSTSTAYMELSSLRSEDTAVYYCARAAWYM
DYWGQGTLVTVSSGGGGSGGGGSGGGGSDIVLTQSPDSLAVSLGE
RATISCKSSQSLLNSRTRENYLAWYQQKPGQPPKLLIYWASTRES
GVPDRFSGSGSGTDFTLTISSLQAEDVAVYYCTQSYNLYTFGQGT
KVEIK.

It will be within the conventional knowledge of the skilled person to select or generate antibodies or antibody fragments specifically directed against CD80 and/or CD86.

The transmembrane domain of the fusion protein of the present invention may be any type of transmembrane domains meeting the structural requirements necessary to maintain the function of the associated extracellular and intracellular domains.

Preferably, the transmembrane domain of the fusion protein of the present invention is selected from the list of transmembrane domains comprised in the protein sequences of the T-cell receptor alpha chain (UniProtKB-P01848), T-cell receptor beta chain (UniProtKB-PODSE2), T-cell receptor zeta chain (UniProtKB-P20963), CD28 (UniProtKB-P10747), CD3 epsilon chain (UniProtKB-P07766), CD3 zeta chain (UniProtKB-P20963), CD45 (UniProtKB-P08575), CD4 (UniProtKB-P01730), CD5 (UniProtKB-P06127), CD8 alpha chain (UniProtKB-P01732), CD8 beta chain (UniProtKB-P10966), CD9 (UniProtKB-P21926), CD16 (UniProtKB-Q9UPY7), CD22 (UniProtKB-P20273). CD33 (UniProtKB-P20138), CD37 (UniProtKB-P11049), FCGR1A (CD64) (UniProtKB-P12314), CD80 (UniProtKB-P33681), CD86 (UniProtKB-P42081), CD134 (UniProtKB-P43489), CD137(UniProtKB-Q07011), CD154 (UniProtKB-P29965), CD200R1 (UniProtKB-Q8TD46), KIR2DS2(UniProtKB-P43631), OX40 (UniProtKB-P23510), CD2 (UniProtKB-P06729), CD27 (UniProtKB - P26842), LFA-1 (CD11a) (UniProtKB-P20701), ICOS (CD278) (UniProtKB-Q9Y6W8), CD137 (4-1BB) (UniProtKB-Q07011). GITR (UniProtKB-Q9Y5U5), CD40 (UniProtKB-P25942), BAFFR (UniProtKB-Q96RJ3), HVEM (UniProtKB-Q92956), SLAMF7 (UniProtKB-Q9NQ25), NKp80(KLRF1) (UniProtKB-Q9NZS2), CD160 (UniProtKB-095971), CD19 (UniProtKB-P15391), IL2R beta (UniProtKB-P14784), IL2R gamma (UniProtKB-P31785), IL7R alpha (UniProtKB-P16871), CD49a (UniProtKB-P56199), IA4 (UniProtKB-Q8IU71), CD49D (UniProtKB-P13612), CD49f (UniProtKB-P23229), ITGAD (CD11d) (UniProtKB-Q13349), ITGAE (CD103) (UniProtKB-P38570), CD11a (UniProtKB-P20701), CD11b (UniProtKB-P11215), CD11c (UniProtKB-P20702), CD29 (UniProtKB-P05556), CD18 (UniProtKB-P05107), ITGB7 (UniProtKB-P26010). TNFR2 (UniProtKB-P20333). DNAM1 (CD226) (UniProtKB-Q15762), SLAMF4 (CD244) (UniProtKB-Q9BZW8), CD84 (UniProtKB-Q9UIB8), CD96 (UniProtKB-P40200), CEACAM1(UniProtKB-P13688), CRTAM (UniProtKB-095727). Ly9 (CD229) (UniProtKB-Q9HBG7), CD160(BY55) (UniProtKB-095971), PSGL1 (UniProtKB-Q14242), CD100 (SEMA4D) (UniProtKB-Q92854), SLAMF6 (UniProtKB-Q96DU3). SLAM (CD150) (UniProtKB-Q13291), BLAME (SLAMF8) (UniProtKB-Q9POV8), SELPLG (CD162) LTBR (UniProtKB-Q14242), PAG-Cbp (UniProtKB-P06241), CTLA-4 (CD152) (UniProtKB-P16410), preferably selected from the list of transmembrane domains comprising the transmembrane domains of CD8 alpha chain (UniProtKB-P01732), CTLA-4 (CD152) (UniProtKB-P16410), CD28 (UniProtKB-P10747), more preferably the transmembrane domain of CTLA-4 (CD152) (UniProtKB-P16410).

The intracellular domain of the fusion protein of the present invention comprising a co-stimulatory polypeptide can comprise a signaling polypeptide or signaling domain. Within the context of the present invention, an intracellular domain comprising a co-stimulatory (or stimulatory) polypeptide may also be designated as intracellular or cytoplasmic signaling domain, intracellular or cytoplasmic activating domain or the like.

Intracellular signaling domains suitable for use in the fusion protein of the present invention may preferably be or comprise an immunoreceptor tyrosine-based activation motif (ITAM)-containing intracellular signaling polypeptide.

An ITAM motif is YX₁X₂L/I, where X₁ and X₂ are independently any amino acid (SEQ ID NO: 7). In certain embodiments, the intracellular signaling domain of the fusion protein comprises 1, 2, 3, 4, or 5 ITAM motifs. In some embodiments, an ITAM motif is repeated twice in an intracellular signaling domain, where the first and second instances of the ITAM motif are separated from one another by 6 to 8 amino acids, e.g., (YX₁X₂L/l)(X₃)n(YX₁X₂L/l) (SEQ ID NO: 8), where n is an integer from 6 to 8, and each of the 6-8 X₃ can be any amino acid. In one embodiment, the intracellular signaling domain of the fusion protein of the present invention comprises 3 ITAM motifs.

A suitable intracellular signaling domain for use in the fusion protein of the present invention can be an ITAM motif-containing portion that is derived from a polypeptide that contains an ITAM motif. For example, a suitable intracellular signaling domain can be an ITAM motif-containing domain from any ITAM motif-containing protein.

Thus, a suitable intracellular signaling domain or co-stimulatory polypeptides need not contain the entire sequence of the entire protein from which it is derived. It will be well within the knowledge of the skilled person to determine functional intracellular signaling domains or co-stimulatory polypeptides comprised in a given entire protein sequence.

Examples of preferred protein sequences comprising ITAM motif-containing polypeptides include, but are not limited to: DAP12 (UniProtKB-043914), FcεRly (Fc epsilon receptor I gamma chain) (UniProtKB-P30273), CD3D (CD3 delta) (UniProtKB-P04234); CD3E (CD3 epsilon) (UniProtKB-P07766). CD3G (CD3 gamma) (UniProtKB-P09693), CD3Z (CD3 zeta) (UniProtKB-P20963), CD79A (antigen receptor complex-associated protein alpha chain) (UniProtKB-P11912) and CD79B (antigen receptor complex-associated protein beta chain) (UniProtKB-P40259).

In a preferred embodiment of the present invention, the co-expressed (CAR) protein comprises an intracellular domain having one or more ITAMs, preferably one of FcεRlγ(ic) and CD3ζ (ic), more preferably CD3ζ (ic), and the fusion protein comprises an intracellular domain selected from the group consisting of 4-1BB (CD137; SEQ ID NO: 3), CD28, ICOS, OX-40, BTLA, CD27, CD30, GITR, Myd88-CD40, KIR2DS2 and HVEM. According to one preferred embodiment, the fusion protein comprises an intracellular domain having one or more ITAMs, preferably one of FcεRlγ (ic) and CD3ζ (ic), more preferably CD3ζ (ic), and the co-expressed (CAR) protein comprises an intracellular domain selected from the group consisting of 4-1BB (CD137; SEQ ID NO: 3), CD28, ICOS, OX-40, BTLA, CD27, CD30, GITR, Myd88-CD40, KIR2DS2 and HVEM.

In a preferred embodiment of the present invention, the intracellular domain comprising a co-stimulatory polypeptide may be selected from the list of intracellular domains or co-stimulatory polypeptides comprised within the protein sequence of CD28 (UniProtKB-P10747), ICOS (UniProtKB-Q9Y6W8), OX-40 (UniProtKB-P43489), BTLA (UniProtKB-Q7Z6A9), CD27 (UniProtKB-P26842), CD30 (UniProtKB-P28908), CD40L (UniProtKB-P29965), GITR (UniProtKB- Q9Y5U5), Myd88 (UniProtKB-Q99836), CD40 (UniProtKB-P25942), KIR2DS2 (UniProtKB-P43631), CD137 (4-1BB) (UniProtKB-Q07011), and HVEM (UniProtKB-Q92956), preferably within the protein sequence of CD137 (4-1BB) (UniProtKB-Q07011), CD28 (UniProtKB-P10747), or OX-40 (UniProtKB-P43489), more preferably of CD137 (4-1BB) (UniProtKB-Q07011).

Within the present invention, the fusion protein may comprise the domains as defined herein and, optionally, additional sequences connecting these domains within the fusion protein. Such linker or hinge sequences for use within CARs or CCRs are commonly known in the art and can easily be determined and identified by a person skilled in the art, so that the specific function of the individual functional domains is not interfered with.

In one preferred embodiment of the present invention, the fusion protein comprises the extracellular domain and the transmembrane domain of CTLA-4 and the intracellular signaling domain of 4-1BB. In a preferred embodiment, the fusion protein comprises the following amino acid sequence (CTLA4ec+in grey, 4-1BB ic in black):

(SEQ ID NO: 9)

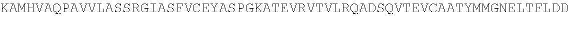

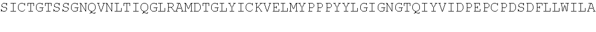

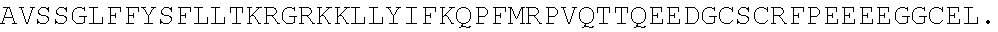

Alternatively preferably, the fusion protein comprises the extracellular domain and the transmembrane domain of CD28 and the intracellular signaling domain of 4-1BB. In a preferred embodiment, the fusion protein comprises the following amino acid sequence (CD28 ec+im in grey, 4-1BB ic in black):

(SEQ ID NO: 10)

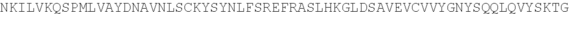

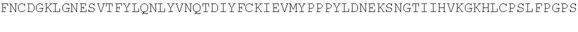

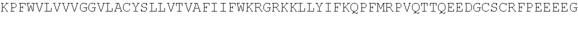

GCEL.

Alternatively preferably, the fusion protein comprises a binding domain directed against CD80, the extracellular domain and the transmembrane domain of CD28 and the intracellular signaling domain of 4-1BB. In a preferred embodiment, the fusion protein comprises the following amino acid sequence (α-CD80 in black, CD28 ec+tm in grey, 4-1BB ic in black, in this order):

(SEQ ID NO: 11)
QLVQSGAEVKKPGASVKVSCKPSGFNIKDYYMHWVRQAPGQGLEWIGWIDPENGNTLYDPKFQ
GKATITADTSTSTAYMELSSLRSEDTAVYYCAREGLFFAYWGQGTLVTVSSGGGGSGGGGSGGG
GSDIQMTQSPSSLSASVGDRVTITCSVSSSISSSNLHWYQQKPGKAPKPLIYGTSNLASGVPSRFS

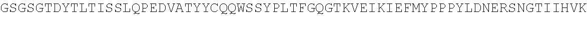

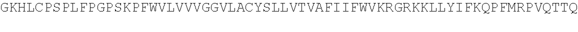

EEDGCSCRFPEEEEGGCEL.

Alternatively preferably, the fusion protein comprises a binding domain directed against CD86, the extracellular domain and the transmembrane domain of CD28 and the intracellular signaling domain of 4-1BB. In a preferred embodiment, the fusion protein comprises the following amino acid sequence (α-CD86 in black, CD28 ec+tm in grey, 4-1BB ic in black, in this order):

(SEQ ID NO: 12)
QVQLVQSGAEVKKPGSSVKVSCKASGYTFTDYAIQWVRQAPGQGLEWIGVINIYYDNTNYNQKFK
GKATMTVDKSTSTAYMELSSLRSEDTAVYYCARAAWYMDYWGQGTLVTVSSGGGGSGGGGSG
GGGSDIVLTQSPDSLGERATISCKSSQSLLNSRTRENYLAWYQQKPGQPPKLLIYWASTRES

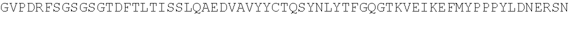

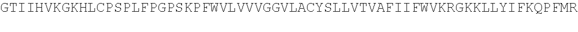

PVQTTQEEDGCSCRFPEEEEGGCEL.

Alternatively preferably, the fusion protein comprises a binding domain directed against CD80, a binding domain directed against CD86, the extracellular domain and the transmembrane domain of CD28 and the intracellular signaling domain of 4-1BB. In a preferred embodiment, the fusion protein comprises the following amino acid sequence (α-CD80 in grey, linker sequence in bold, α-CD86 in black, CC28 on ec+tm in grey, 4-18B ic in black, in this order):

(SEQ ID NO: 13)

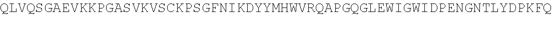

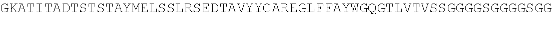

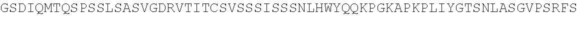

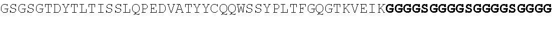

SQVQLVQSGAEVKKPGSSVKVSCKASGYTFTDYAIQWVRQAPGQGLEWIGVINIYYDNTNYNQKF
KGKATMTVDKSTSSTAYMELSSLRSEDTAVYYCARAAWYMDYWGQGTLVTVSSGGGGSGGGGS
GGGGSDIVLTQSPDSLAVSLGERATISCKSSQSLLNSSRTRENYLAWYQQKPGQPPKLLIYWASTR

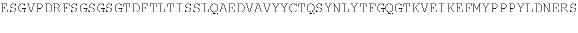

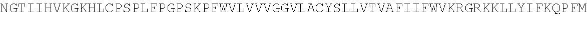

RPVQTTQEEDGCSCRFPEEEEGGCEL.

Additional sequences such as leader sequences (e.g. lg kappa light chain leader sequences such as MDFQVQIFSFLLISASVIMSR (SEQ ID NO: 14)) or others may also be present in the fusion protein of the present invention.

In one specific embodiment, the fusion protein of the present invention may be expressed as a combined protein construct with a given CAR to be expressed by the same cell, wherein the construct comprises a protein cleavage site between the two proteins, preferably a self-cleaving peptide, more preferably a 2A self-cleaving peptide, particularly preferably of the sequence GSGATNFSLLKQAGDVEENPGP (SEQ ID NO: 15).

According to one particular embodiment, the fusion protein is co-expressed together with the respective CAR, preferably comprising respective leader sequences and/or a self-cleaving peptide between the two protein entities, more preferably of the following general structure:

leader sequence (e.g. L kappa leader sequence)—extracellular domain directed against cell surface proteins associated with tumors (e.g. CD19, CD20, CD22)—optionally extracellular domain of CD80α hinge-transmembrane domain (e.g. of CD8a hinge)-intracellular signaling domain (e.g. of CD3ζ) —self-cleaving peptide (e.g. P2A self-cleaving peptide)-leader sequence (e.g. L kappa leader sequence)—extracellular domain directed against CD80 and/or CD86 (e.g. of CTLA-4)—transmembrane domain (e.g. of CTLA-4)-intracellular signaling domain (e.g. of 4-1BB).

According to a specific embodiment, the fusion protein co-expressed together with the respective CAR has the following amino acid sequence:

(SEQ ID NO: 16)
MDFQVQIFSFLLISASVIMSRDIQMTQTTSSLSASLGDRVTISCR
ASQDISKYLNWYQQKPDGTVKLLIYHTSRLHSGVPSRFSGSGSGT
DYSLTISNLEREDIATYFCQQGNTLPYTFGGGTKLEITGGGGSGG
GGSGGGGSEVKLQESGPGLVAPSQSLSVTCTVSGVSLPDYGVSWI
RQPPRKGLEWLGVIWGSETTYYNSALKSRLTIIKDNSKSQVFLKM
NSLQTDDTAIYYCAKHYYYGGSYAMDYWGQGTSVTVSSTTTPAPR
PPTPAPTIASQPLSLRPEACRPAAGGAVHTRGLDFACDIYIWAPL
AGTCGVLLLSLVITLYCRVKFSRSADAPAYKQGQNQLYNELNLGR
REEYDVLDKRRGRDPEMGGKPRRKNPQEGLYNELQKDKMAEAYSE
IGMKGERRRGKGHDGLYQGLSTATKDTYDALHMQALPPRGSGATN
FSLLKQAGDVEENPGPMDFQVQIFSFLLISASVIMSRKAMHVAQP
AVVLASSRGIASFVCEYASPGKATEVRVTVLRQADSQVTEVCAAT
YMMGNELTFLDDSICTGTSSGNQVNLTIQGLRAMDTGLYICKVEL
MYPPPYYLGIGNGTQIYVIDPEPCPDSDFLLWILAAVSSGLFFYS
FLLTKRGRKKLLYIFKQPFMRPVQTTQEEDGCSCRFPEEEEGGCE
L.

According to the present invention, any functional domain as mentioned herein (e.g. extracellular domain, transmembrane domain and/or intracellular signaling domain in case of the fusion protein of the present invention) may be a domain which is functionally equivalent to the domains individually recited herein.

For example, an extracellular domain which does not have exactly the same sequence as the CTLA-4 extracellular domain but maintains specific affinity to CD80 and/or CD86 may still be suitable as the extracellular domain of the fusion protein according to the present invention. In this regard, it is pointed out that the specific function of such domains with different sequences can easily be identified and assessed by a person skilled in the art.

Therefore, according to one embodiment of the present invention, protein sequences form part of the invention as the individual domains of the proteins of the present invention which consist of or comprise a protein sequence being at least 80% identical to the referenced protein sequences disclosed herein, preferably at least 85% identical, more preferably at least 90% identical, even more preferably at least 95% identical, even more preferably at least 98% identical, particularly preferably at least 99% identical.

The determination of percent identity between two sequences is accomplished according to the present invention by using the mathematical algorithm of Karlin and Altschul (Proc. Natl. Acad. Sci. USA (1993) 90:5873-5877). Such an algorithm is the basis of the BLASTN and BLASTP programs of Altschul et al. (J. Mol. Biol. (1990) 215: 403-410). BLAST nucleotide searches are performed with the BLASTN program. To obtain gapped alignments for comparative purposes, Gapped BLAST is utilized as described by Altschul et al. (Nucleic Acids Res. (1997) 25:3389-3402). When utilizing BLAST and Gapped BLAST programs, the default parameters of the respective programs are used.

According to one specific embodiment of the present invention, protein sequences forming part of the present invention as defined above by a given percent identity to the individualized protein sequences are those that maintain the function of the respective proteins.

According to one preferred embodiment of the present invention, the polypeptide with specific affinity to CD80 and/or CD86 has an amino acid sequence which is at least 80% identical to the amino acid sequence of the extracellular domain of human CTLA-4 (SEQ ID NO: 1) or an amino acid sequence which is at least 80% identical to the amino acid sequence of the extracellular domain of human CD28 (SEQ ID NO: 2), or an amino acid sequence which is at least 80% identical to the amino acid sequence of the CD86-binding domain of the anti-CD86 antibody commonly referred to in the art as cione hu3D1 (SEQ ID NO: 5), preferably wherein the polypeptide with specific affinity to CD80 and/or CD86 has an amino acid sequence which is at least 80% identical to the amino acid sequence of the extracellular domain of human CTLA-4 (SEQ ID NO: 1)

According to another preferred embodiment of the present invention, the transmembrane domain is suitable for insertion and anchoring of the fusion protein in the cell membrane of a mammalian cell, preferably wherein the transmembrane domain comprises the transmembrane region of one or more of the alpha, beta or zeta chain of the T-cell receptor, CTLA-4, CD28, CD3 epsilon, CD45, CD4, CD5,CD8 (e.g., CD8 alpha, CD8 beta), CD9, CD16, CD22, CD33, CD37, CD64, CD80, CD86, CD134, CD137, CD154, CD200, KIRDS2, 0X40, CD2, CD27, LFA-1 (CDIIa, CD18). ICOS (CD278). 4-1BB (CD137), GITR, CD40, BAFFR, HVEM (LIGHTR), SLAMF7, NKp80 (KLRF1), CD160, CD19, IL2R beta, IL2R gamma, IL7R .alpha., VLA1, ITGA1, CD49a, ITGA4, IA4, CD49D, ITGA6, VLA-6, CD49f, ITGAD, CDIId, ITGAE, CD103, ITGAL, CDIla, LFA-1, ITGAM, CDIIb, ITGAX, CDIIc, ITGB1, CD29,ITGB2, CD18, LFA-1, ITGB7, TNFR2, DNAM1 (CD226), SFAMF4 (CD244, 2B4), CD84, CD96 (Tactile), CEACAM1, CRTAM. Ly9 (CD229). CD160 (BY55), PSGL1, CD100 (SEMA4D), SLAMF6(NTB-A, Ly108), SEAM (SLAMF1, CD150, IPO-3), BLAME (SLAMF8), SELPLG (CD162), LTBR, and PAG/Cbp, or an amino acid sequence which is at least 80% identical thereto, preferably wherein the transmembrane domain the transmembrane region of human CTLA-4 (FLLWILAAVSSGLFFYSFLLT: SEQ ID NO: 17), or an amino acid sequence which is at least 80% identical thereto.

The inclusion of a costimulatory domain, such as the 4-IBB (CD 137) costimulatory domain in the intracellular region of the fusion protein of the present invention enables the T cell to receive co-stimulatory signals which can guide and control the specific T cell response.

According to a preferred embodiment of the present invention, the intracellular domain is able to cause costimulatory signals to the T cell. Preferably, the intracellular domain of the fusion protein of the present invention comprises the intracellular domain or a (co-)stimulatory polypeptide of one or more of human 4-1BB (CD137; SEQ ID NO. 3), CD3ζ(SEQ ID NO: 25), CD28, ICOS, OX-40, BTLA, CD27, CD30, GITR, Myd88-CD40, KIR2DS2 and HVEM, or an amino acid sequence which is at least 80% identical thereto, more preferably the intracellular domain of the fusion protein of the present invention comprises the intracellular domain or a co-stimulatory polypeptide of human 4-1BB (CD137; (SEQ ID NO: 3)), or an amino acid sequence which is at least 80% identical thereto.

In a preferred embodiment, the fusion protein of the present invention comprises an extracellular domain comprising the extracellular domain of human CTLA-4 (SEQ ID NO: 1), or an amino acid sequence which is at least 80% identical thereto, and the intracellular domain comprises a co-stimulatory peptide comprising the intracellular domain of human 4-1BB (CD137; (SEQ ID NO: 3)), or an amino acid sequence which is at least 80% identical thereto.

More preferably, the fusion protein comprises the following amino acid sequence of CTLA-4 (ec+tm) fused to 4-1BB (ic):

(SEQ ID NO: 18)
KAMHVAQPAVVLASSRGIASFVCEYASPGKATEVRVTVLRQADSQ
VTEVCAATYMMGNELTFLDDSICTGTSSGNQVNLTIQGLRAMDTG
LYICKVELMYPPPYYLGIGNGTQIYVIDPEPCPDSDFLLWILAAV
SSGLFFYSFLLTKRGRKKLLYIFKQPFMRPVQTTQEEDGCSCRFP
EEEEGGCEL.

The fusion protein of the present invention may generally be used in combination with chimeric antigen receptor (CAR) targeting cell surface proteins associated with tumors. In particular, the fusion protein may preferably be used in combination with CARs directed against any of CD19, CD20 or CD22. Based on the data provided herein, it is plausible and credible that the present invention will be functional and leading to advantageous results when used with CARs against CD19, CD20 or CD22, or any other protein known to be expressed on the surface of tumor cells and/or as tumor marker in this context.

In a particular embodiment, the fusion protein of the present invention may be used in combination with a CAR directed against CD19. In another particular embodiment, the fusion protein of the present invention may be used in combination with a CAR directed against CD20. In yet another particular embodiment, the fusion protein of the present invention may be used in combination with a CAR directed against CD22.

In another aspect, the present invention further relates to a nucleic acid molecule encoding the fusion protein as provided and disclosed herein. The present Invention also relates to nucleic acid molecules which only encode parts of the fusion protein described and provided herein, e.g., which encode only the extracellular domain, the transmembrane domain, and/or the intracellular domain of the fusion protein.

As used herein, unless specifically defined otherwise, the term “nucleic acid” or “nucleic acid molecule” is used synonymously with “oligonucleotide”, “nucleic acid strand”, “polynucleotide”, or the like, and means a polymer comprising one, two, or more nucleotides.

The term “nucleic acid molecule” relates to the sequence of bases comprising purine-and pyrimidine bases which are comprised by polynucleotides, whereby said bases represent the primary structure of a nucleic acid molecule.

Herein, the term “nucleic acid molecule” includes all kinds of nucleic acid, including DNA, cDNA, genomic DNA, RNA, synthetic forms of DNA and mixed polymers comprising two or more of these molecules, and preferably relates to DNA and cDNA. As readily understood by those of skill in the art, the nucleic acid sequences provided herein represent sequences of DNA and also comprise corresponding RNA sequences where T is replaced by U.

The term “nucleic acid molecule” generally comprises sense and antisense strands. “Nucleic acid molecule” may further comprise non-natural or derivatized nucleotide bases as well as natural or artificial nucleotide analogues, e.g., in order to protect the nucleic acid molecule against endo- and/or exonucleases as will be readily appreciated by those skilled in the art.

In yet another aspect, the present invention further relates to a vector comprising the nucleic acid molecule described and provided herein.

The term “vector” as used herein generally comprises all kinds of linear or circular nucleic acid molecules which can replicate autonomously in a suitable host cell. Such vectors comprise, but are not limited to, plasmids, cosmids, phages, virus (e.g., adeno-, adeno-associated-, lenti-, or preferably retroviral vectors), and other vectors or shuttles known in the art which are suitable to carry and transfer genes into host cells in order to allow stable or transient translation and constitutive or conditional expression of the fusion protein of the present invention in the host cell.

The vector is usually not integrated into the cell genome, but may also be integrated. Vectors according to the present invention which comprise nucleic acid molecules as described and provided herein preferably allow stable expression of the fusion protein of the present Invention in the host cell (expression vector).

Vectors of the present invention may further comprise marker genes, promoter and/or enhancer sequences (operably linked to the nucleic acid molecule of the present invention), replication origins suitable for the respective host cell, restriction sites, multiple cloning sites, labels and further functional units as known in the art.

The vectors may inter alia be transferred into host cells via a shuttle such as a virus (which may itself be considered a vector), or be transformed in naked form or transduced into host cells. The vector is preferably adapted to be suitable for the respective host cell where it is to be transformed or transduced into.

The skilled person will readily understand that different host cells will require different kinds of vectors. For example, the vector (plasmid) pGEM is a suitable vector for transformation into bacterial cells, while the retroviral vector pMP71 is suitable for transduction into eukaryotic cells (e.g., T cells).

In one embodiment of the present invention, the vector of the present invention is a viral vector, e.g., a retroviral or lentiviral vector, e.g., a retroviral vector. Examples for suitable retroviral vectors are known in the art and include, e.g., pMP71-PRE (Leisegang, K Mol Med (2008), 86(5): 573-583), SAMEN CMV/SRa, LZRS-id3-IHRES (Heemskerk et al., J. Exp. Med. 186 (1997), 1597-1602), FeLV (Neil et al., Nature 308 (1984), 814-820), SAX (Kantoff et al., Proc. Natl. Acad. Sci. USA 83 (1986), 6563-6567), pDOL (Desiderio, J. Exp. Med. 167 (1988), 372-388), N2 (Kasid et al., Proc. Natl. Acad. Sci. USA 87 (1990), 473-477). LNL6 (Tiberghien et al., Blood 84 (1994), 1333-1341), pZipNEO (Chen et al., J. Immunol. 153 (1994), 3630-3638), LASN (Mullen et al., Hum. Gene Ther. 7 (1996), 1123-1129), pGIXsNa (Taylor et al., J. Exp. Med. 184 (1996), 2031-2036), LCNX (Sun et al., Hum. Gene Ther. 8 (1997), 1041-1048), SFG (Gallardo et al., Blood 90 (1997), LXSN (Sun et al., Hum. Gene Ther. 8 (1997), 1041-1048), SFG (Gallardo et al., Blood 90 (1997), 952-957), HMB-Hb-Hu (Vieillard et al., Proc. Natl. Acad. Sci. USA 94 (1997), 11595-11600), pMV7 (Cochlovius et al., Cancer Immunol. Immunother. 46 (1998), 61-66), pSTITCH (Weitjens et al., Gene Ther 5 (1998), 1195-1203), pLZR (Yang et al., Hum. Gene Ther. 10 (1999), 123-132), pBAG (Wu et al., Hum. Gene Ther. 10(1999), 977-982), rKat.43.267bn (Gilham et al., J. Immunother. 25 (2002), 139-151), pLGSN (Engels et al., Hum. Gene Ther. 14 (2003), 1155-1168), pMP71 (Engels et al., Hum. Gene Ther. 14 (2003), 1155-1168), pGCSAM (Morgan et al, J. Immunol 171 (2003), 3287-3295), pMSGV (Zhao et al., J. Immunol. 174 (2005), 4415-4423), or pMX (de Witte et al., J. Immunol. 181 (2008), 5128-5136).

In a further aspect, the present invention relates to a host cell comprising the nucleic acid molecule or the vector as described and provided herein. In one embodiment, the host cell of the present invention is transduced or transformed with the nucleic acid molecule or the vector as described and provided herein.

Generally, as used herein unless specifically defined otherwise, the terms “transduced” or “transformed” (as well as “transduction” or “transformation”) or the like may be used interchangeably and generally mean any kind of transfer of a nucleic acid molecule and/or vector into a host cell, regardless of the kind of host cell and regardless of the way of transfer (e.g., (chemical) transformation, (viral) transduction, electroporation, transfection, etc.).

The nucleic acid molecule and/or the vector may be stably integrated into the genome of the host cell, or be extrachromosomal (i.e. transient expression). Examples for suitable methods for achieving transient expression in a host cell are known in the art and comprise mRNA transfection. In one embodiment, the host cell is transduced with the nucleic acid molecule and/or the vector. In another embodiment, the nucleic acid molecule and/or the vector is stably integrated into the genome of the host cell.

The host cell described and provided as part of the present invention comprising the nucleic acid molecule or the vector as described and provided herein is preferably able to stably or transiently (e.g., stably) express (either constitutively or conditionally) the fusion protein of the present invention.

The host cell may generally be transduced or transformed by any method with any suitable nucleic acid molecule or vector. In one embodiment, the host cell is transduced with a retroviral or lentiviral (e.g., retroviral) vector comprising a nucleic acid molecule encoding the fusion protein of the present invention er parts thereof (e.g., extracellular domain, transmembrane domain, and/or intracellular domain) as described above.

In one embodiment, the host cell of the present invention is transduced with a retroviral vector comprising a nucleic acid molecule encoding the fusion protein of the present invention or parts thereof (e.g., extracellular domain, transmembrane domain, and/or intracellular domain) as described above and stably or transiently expresses (either constitutively or conditionally) the fusion protein or one or more parts thereof.

Preferably, the host cell then stably or transiently expresses the fusion protein in its membrane, with the extracellular domain of the fusion protein of the present invention directed towards the exterior of the host cell, the transmembrane domain being (largely) embedded in the host cell membrane, and the intracellular domain extending into the cytoplasm of the host cell.

Within the present invention, the host cell comprising the nucleic acid molecule or the vector as described and provided herein relates to a genetically modified cell which was transduced or transformed with the nucleic acid molecule or vector, or wherein said nucleic acid molecule or vector was otherwise introduced into the host cell.

As already mentioned, the host cell of the present invention may be a cell which transiently or stably expresses the fusion protein of the present invention. For example, the nucleic acid molecule encoding the fusion protein of the present invention can be stably integrated into the genome of the cell by retroviral or lentiviral (e.g., retroviral) transduction.

The host cell or transduced cell of the present invention may be, e.g., CD8⁺ T Cells, CD4⁺ T cells, TCR such as (but not limited to) TCR-T58 or TCR-D115 T Cells, double-negative α/β T cells, NK (natural killer) cells, γō T cells, macrophages, dendritic cells, as well as cells suitable to store and/or reproduce the nucleic acid molecule or vector of the present invention, including bacterial cells (e.g., E. coli) and further eukaryotic cells. The cells may be autologous or non-autologous, but are preferably autologous. Also, the cells may be allogeneic or non-allogeneic as readily apparent for the skilled person.

In one embodiment, the host cell of the present Invention is a T cell, preferably a CD8⁺ T cell.

The host cell of the present invention may be transduced with a nucleic acid molecule or a vector encoding the fusion protein as described and provided herein. Preferably, the host cell provided and described herein may be co-transduced with further nucleic acid molecules, e.g. with a nucleic acid molecule encoding a T cell receptor (TCR) or a chimeric antigen receptor (CAR), e.g. as also described herein.

Such co-transduction (or other method for introducing nucleic acid molecules into cells as described and exemplified herein) is known in the art and also described and exemplified herein.

In one embodiment, the host cell stably or transiently expressing the fusion protein of the present invention additionally stably or transiently co-expresses a (co-expressed CAR) protein comprising an extracellular domain with specific affinity to CD19 and an intracellular co-stimulatory domain, preferably wherein the extracellular domain with specific affinity to CD19 comprises at least an antigen-binding fragment of an anti-CD19 antibody, more preferably wherein the extracellular domain with specific affinity to CD19 comprises an anti-CD19 scFv.

In another embodiment, the host cell stably or transiently expressing the fusion protein of the present invention additionally stably or transiently co-expresses a (co-expressed CAR) protein comprising an extracellular domain with specific affinity to CD20 and an intracellular co-stimulatory domain, preferably wherein the extracellular domain with specific affinity to CD20 comprises at least an antigen-binding fragment of an anti-CD20 antibody, more preferably wherein the extracellular domain with specific affinity to CD20 comprises an anti-CD20 scFv.

In all embodiments described herein as including stably or transiently expressing a protein, stable expression of the protein is preferred in any context.

Preferably, said co-expressed (CAR) protein comprises as an intracellular costimulatory domain an amino acid sequence of CD3ζ((SEQ ID NO: 25), or an amino acid sequence which is at least 80% identical thereto.

According to one embodiment, the co-expressed (CAR) protein comprises the binding domain of an anti-CD19 antibody as an extracellular domain, preferably an anti-CD19 scFv, more preferably the anti-CD19 scFv commonly referred to in the art as clone FMC63 as an extracellular domain. The sequence of said anti-CD19 scFv is as follows:

(SEQ ID NO: 19)
DIQMTQTTSSLSASLGDRVTISCRASQDISKYLNWYQQKPDGTVK
LLIYHTSRLHSGVPSRFSGSGSGTDYSLTISNLEQEDIATYFCQQ
GNTLPYTFGGGTKLEITGGGGSGGGGSGGGGSEVKLQESGPGLVA
PSQSLSVTCTVSGVSLPDYGVSWIRQPPRKGLEWLGVIWGSETTY
YNSALKSRLTIIKDNSKSQVFLKMNSLQTDDTAIYYCAKHYYYGG
SYAMDYWGQGTSVTVSS.

Further, the co-expressed (CAR) protein may alternatively or additionally comprise the binding domain of an anti-CD20 antibody as an extracellular domain, preferably the binding domain of the anti-CD20 antibody commonly referred to in the art as clone 2H7. The sequence of said binding domain is as follows:

(SEQ ID NO: 20)
MAQVKLQESGAELVKPGASVKMSCKASGYTFTSYNMHWVKQTPGQ
GLEWIGAIYPGNGDTSYNQKFKGKATLTADKSSSTAYMQLSSLTS
EDSADYYCARSNYYGSSYWFFDWWVGQGTTVTVSSGGGGSGGGGG
GGGSDIELTQSPTILSASPGEKVTMTCRASSSVNYMDWYQKKPGS
SPKPWIYATSNLASGVPARFSGSGSGTSYSLTISRVEAEDAATYY
CQQWSFNPPTFGGGTKLEIKRAAA.

Also, the co-expressed (CAR) protein may alternatively or additionally comprise the binding domain of an anti-CD22 antibody as an extracellular domain, preferably the binding domain of the anti-CD22antibody commonly referred to in the art as clone m971. The sequence of said binding domain is as follows:

(SEQ ID NO: 21)
QVQLQQSGPGLVKPSQTLSLTCAISGDSVSSNSAAWNWIRQSPSR
GLEWLGRTYYRSKWYNDYAVSVKSRITINPDTSKNQFSLQLNSVT
PEDTAVYYCAREVTGDLEDAFDIWGQGTMVTVSSGGGGSGGGGSG
GGGSDIQMTQSPSSLSASVGDRVTITCRASQTIWSYLNWYQQRPG
KAPNLLIYAASSLQSGVPSRFSGRGSGTDFTLTISSLQAEDFATY
YCQQSYSIPQTFGQGTKLEIK

In one embodiment, the co-expressed (CAR) protein may comprise the extracellular and transmembrane domain of CD8 alpha together with the intracellular domain of CD3ζ, preferably having the amino acid sequence as follows:

(SEQ ID NO: 22)
TTTPAPRPPTPAPTIASQPLSLRPEACRPAAGGAVHTRGLDFACD
IYIWAPLAGTCGVLLLSLVITLYCRVKFSRSADAPAYKQGQNQLY
NELNLGRREEYDVLDKRRGRDPEMGGKPRRKNPQEGLYNELQKDK
MAEAYSEIGMKGERRRGKGHDGLYQGLSTATKDTYDALHMQALPP
R

In one specific embodiment, the co-expressed (CAR) protein may comprise an anti-CD19 scFv together with the extracellular and transmembrane domain of CD8 alpha and the intracellular domain of CD3ζ, preferably having the amino acid sequence as follows:

(SEQ ID NO: 23)
DIQMTQTTSSLSASLGDRVTISCRASQDISKYLNWYQQKPDGTVK
LLIYHTSRLHSGVPSRFSGSGSGTDYSLTISNLEQEDIATYFCQQ
GNTLPYTFGGGTKLEITGGGGSGGGGSGGGGSEVKLQESGPGLVA
PSQSLSVTCTVSGVSLPDYGVSWIRQPPRKGLEWLGVIWGSETTY
YNSALKSRLTIIKDNSKSQVFLKMNSLQTDDTAIYYCAKHYYYGG
SYAMDYWGQGTSVTVSSTTTPAPRPPTPAPTIASQPLSLRPEACR
PAAGGAVHTRGLDFACDIYIWAPLAGTCGVLLLSLVITLYCRVKF
SRSADAPAYKQGQNQLYNELNLGRREEYDVLDKRRGRDPEMGGKP
RRKNPQEGLYNELQKDKMAEAYSEIGMKGERRRGKGHDGLYQGLS
TATKDTYDALHMQALPPR

In a particular embodiment, the CD19 CAR of the generally known product Kymriah® or Tisagenlecleucel may be used within the present invention.

Alternatively, the co-expressed (CAR) protein may comprise an anti-CD20 binding domain and an anti-CD19 scFv together with the extracellular and transmembrane domain of CD8 alpha and the intracellular domain of CD3ζ, preferably having the amino acid sequence as follows:

(SEQ ID NO: 24)
MAQVKLQESGAELVKPGASVKMSCKASGYTFTSYNMHWVKQTPGQ
GLEWIGAIYPGNGDTSYNQKFKGKATLTADKSSSTAYMQLSSLTS
EDSADYYCARSNYYGSSYWFFDVWGQGTTVTVSSGGGGSGGGGSG
GGGSDIELTQSPTILSASPGEKVTMTCRASSSVNYMDWYQKKPGS
SPKPWIYATSNLASGVPARFSGSGSGTSYSLTISRVEAEDAATYY
CQQWSFNPPTFGGGTKLEIKRAAAGGGGSGGGGSGGGGSGGGGSD
IQMTQTTSSLSASLGDRVTISCRASQDISKYLNWYQQKPDGTVKL
LIYHTSRLHSGVPSRFSGSGSGTDYSLTISNLEQEDIATYFCQQG
NTLPYTFGGGTKLEITGSTSGSGKPGSGEGSTKGEVKLQESGPGL
VAPSQSLSVTCTVSGVSLPDYGVSWIRQPPRKGLEWLGVIWGSET
TYYNSALKSRLTIIKDNSKSQVFLKMNSLQTDDTAIYYCAKHYYY
GGSYAMDYWGQGTSVTVSSTTTPAPRPPTPAPTIASQPLSLRPEA
CRPAAGGAVHTRGLDFACDIYIWAPLAGTCGVLLLSLVITLYCRV
KFSRSADAPAYKQGQNQLYNELNLGRREEYDVLDKRRGRDPEMGG
KPRRKNPQEGLYNELQKDKMAEAYSEIGMKGERRRGKGHDGLYQG
LSTATKDTYDALHMQALPPR.

In one aspect, the intracellular costimulatory domain of the fusion protein or of the co-expressed (CAR) protein comprises an amino acid sequence of the intracellular domain of CD3ζ, preferably having the amino acid sequence as follows:

RVKFSRSADAPAYKQGQNQLYNELNLGRREEYDVLDKRRGRDPEMGGKPRRKNPQEGLYNELQ KDKMAEAYSEIGMKGERRRGKGHDGLYQGLSTATKDTYDALHMQALPPR (SEQ ID NO: 25) or an amino acid sequence which is at least 80% identical to the amino acid sequence.

In one aspect, the present invention also relates to a method of preparing a host cell of the present invention as described and provided herein, said method comprising transducing (or transforming) a host cell as described above with a nucleic acid molecule or a vector as described and provided herein, cultivating the transduced (or transformed) host cell in a suitable medium allowing growth of the cell and expression of the fusion protein encoded by said nucleic acid molecule or said vector; and collecting the host cells from the medium.

In a preferred embodiment of the present invention, the host cell is transduced or transformed outside the human body. Methods for obtaining, isolating and culturing cells (e.g., T cells such as CD8⁺ T cells, CD4⁺ T cells, TCR such as (but not limited to) TCR-T58 or TCR-D115 T Cells) from donors (e.g., human donors) are known in the art and comprise inter alia blood draw or bone marrow removal.

In accordance with the method of the present invention, the host cell may be transduced or transformed or otherwise be provided with a nucleic acid molecule or a vector as described and provided herein by any method known in the art. Such methods comprise, inter alia, (chemical) transformation, (viral) transduction, electroporation, transfection, and the like. In one embodiment, the host cell is transduced with a retroviral vector.

The present invention also relates to a host cell obtainable by the preparation method provided herein. In another aspect, the present invention further relates to a pharmaceutical composition comprising a fusion protein, a nucleic acid molecule, a vector, and/or a host cell as described and provided by the present invention. Such pharmaceutical composition is suitable to be administered to a patient (preferably a human patient), particularly to the donor of the host cells as described above.

The pharmaceutical composition of the present invention may further comprise a pharmaceutically acceptable carrier and further components, for galenic purposes.

The fusion protein, the nucleic acid molecule, the vector, the host cell and the pharmaceutical composition are each particularly advantageous for use as a medicament, preferably for use in treating diseases and disorders associated with the co-expression of CD19 (or other tumor markers such as, but not limited to, CD20 and CD22) with CD80 and/or CD86, more preferably for use in the treatment of B cell lymphoma, even more preferably for the treatment of non-Hodgkin lymphoma selected from the group comprising marginal zone B cell lymphoma (MZL), mucosa-associated lymphatic tissue lymphoma (MALT), small lymphocytic lymphoma/chronic lymphocytic leukemia (SLL/CLL), mantle cell lymphoma (MCL), Burkitt's lymphoma, lymphoplasmacytic lymphoma, Waldenström's macroglobulinemia, nodal marginal zone B cell lymphoma (NMZL), splenic marginal zone lymphoma (SMZL), and diffuse large B cell lymphoma (DLBCL), particularly preferably for the treatment of diffuse large B cell lymphoma (DLBCL).

Other and additional diseases and disorders which may be treated using the present invention are apparent to the skilled person and comprise particularly (but not limited to) different types of cancer such as lung cancer, gastric cancer, renal cell cancer, color cancer, breast cancer, ovarian cancer, urothelial cancer, melanoma, pancreatic cancer, myeloma, Hodgkin's lymphoma, retinoblastoma, leukemia, cervical cancer, esophageal cancer, glioma, non-Hodgkin's lymphoma, hepatocellular cancer, oral cancer, and others.

Additionally, the present invention also relates to methods for treating a disease or disorder as recited above by administering a pharmaceutical composition comprising a fusion protein, a nucleic acid molecule, a vector, and/or a host cell as described and provided by the present invention. The present invention further relates to methods for treating a disease or disorder as recited above by using a fusion protein, a nucleic acid molecule, a vector, and/or a host cell as described and provided by the present invention.

Ultimately, the present invention also relates to a kit or kit-in-parts comprising a fusion protein, a nucleic acid molecule, a vector, a host cell or a pharmaceutical composition as described and provided as part of the present invention, and a container.

All embodiments of the present invention as disclosed and described herein are deemed to be combinable in any combination, unless the skilled person considers such a combination to not make any technical sense. The invention is now further explained by individual examples which are intended to illustrate but not to limit the present invention.

### EXAMPLES

### PBMC Isolation and T cell Activation

Peripheral blood mononuclear cells were harvested from blood donor buffy coats via density gradient isolation. Buffy coats were carefully pipetted onto STEMCELL Technologies Lymphoprep and centrifuged at 600×g for 30 minutes. The resulting mononuclear cell layer was extracted, washed 4 times in PBS and up to 1e9 cells put into culture in ThermoFisher RPMI 1640 medium with 10% FCS, Pen/Strep and HEPES buffer. T cells were stimulated with IL-2 (1000 U/ml), anti-human CD3, clone OKT3 (200 ng/ml) and anti-human CD28 (100 ng/ml), clone 15E8 for two days before transduction. Primary mouse cells were gathered by singularizing the spleen of C57BL/6N (Black 6; RRID:IMSR_JAX:000664) mice and isolating either B cells with a MojoSort Mouse Pan B Cell Isolation Kit II (BioLegend Cat #480087) or T cells with a MojoSort Mouse CD T cell Isolation Kit (BioLegend Cat #480031) according to the manufacturer's instructions. Murine B cells were cultured in RPMI 1640 supplemented with 10% FCS and Pen/Strep, murine T cells were cultured in X-Vivo 15 supplemented with 5% FCS and stimulated with 200 ng mL⁻¹ anti-CD3, 100 ng mL⁻¹, 1000 U mL⁻¹ IL-2 and 10 ng mL⁻¹ IL-15 for three days prior to transduction.

### Vector Transfection and Transduction

CAR and CAR/CCR expression was induced by retroviral transduction. Two 10 cm plates with monolayer of HEK293t cells was transfected at 50-70% confluence using 20 μl Polyplus PeiPro transfection reagent with 10 μg vector DNA and 5 μg each of GAL-V and M-MuLV helper plasmids in 500 μl RPMI 1640. Transfection reagents were added onto the HEK293t cells cultured in 9 ml of RPMI 1640 with 10% FCS, Pen/Strep and HEPES buffer.

The culture medium containing transduction viruses was harvested after 10-24 hours and added to 1,6e7 PBMC-derived T cells in a plate coated with poly-D-Lysin, centrifuged at 450 g for 90 minutes and cultured overnight. The process was repeated a second time with the same cells to improve transduction yields. During transduction T cells were kept stimulated overnight with IL-2 (1000 U/ml) after the first, and IL-2 (500 U/ml) after the second run.

### Fluorescence Based In-Vitro Cytotoxicity Assays

Cytolytic activity of studied CAR and CAR/CCR T cells was evaluated by measuring fluorescence levels over time in cocultures of T cells and eGFP-expressing tumor cell lines using the HIDEX Sense microplate reader platform with the compatible digital atmospheric control. Cytolytic activity is given as inverse relative fluorescence level increase compared to measurements of only tumor containing wells, calculated using the formula:

$${CyTox}_{i} = 1 - \frac{\frac{( {Fl}_{i} - {Med}_{i} )}{( {Fl}_{0} - {Med}_{0} )}}{\frac{( {Tu}_{i} - {Med}_{i} )}{( {Tu}_{0} - {Med}_{0} )}}$$

Where Fl=Fluorescence measurement in co-culture wells, Tu=Fluorescence measurement in wells containing only tumor cells and Med=Fluorescence measurements in wells containing only the cultivation medium. Assay wells contained 200 μl RPMI 1640 with 10% FCS, Pen/Strep and HEPES, tumor cells and CAR-T cells in different ratios, normalized to total CAR-T cell numbers and total cell numbers by adding Mock-transduced T cells from the same donor.

### Flow Cytometry Based In-Vitro Cytotoxicity Assays

Cytotoxic effect on primary human B cells was investigated after co-culture with CAR/CCR and CAR (2nd gen) T cells for 18 hours with an effector-target ratio of 1:10 at 37° C. in a humidity-controlled environment via flow cytometry. Samples were stained with anti-CD3 APC (Miltenyi Biotec Cat #130-113-125; RRID:AB_2725953), FITC (Miltenyi anti-CD19 Biotec Cat #130-113-645; RRID:AB_2726198) and 7-AAD (BioLegend Cat #420404), washed twice and analyzed.

Cytotoxic effect on primary mouse B cells by mouse T cells equipped with murine CAR/CCR and mCAR (2nd Gen) constructs in experiments was detected by pre-staining the target cells with CellTrace™ Violet Proliferation Kit before adding the effector cells for 24-48 hours. Cells were then detected using the MACSQuant X flow cytometer (Miltenyi Biotec, Bergisch-Gladbach, Germany) and absolute counts of CellTrace™ Violet positive cells plotted for comparison between constructs.

### Cytokine Level Detection in Supernatant via ELISA

Concentration of secreted cytokines in assay supernatants was measured using Sandwich-ELISA technology. Nunc MaxiSorp™ 96-well plates were coated with capture antibodies (detailed below), washed, blocked, and incubated with 50 μl of assay supernatant at 4° C. overnight. PBS dilution of supernatants was applied where necessary. Samples were then discarded and capture antibodies and secondary enzyme-linked antibodies incubated successively following 4 PBS-Tween (0.05% v/v) washing steps between each step.

Detection was performed with TMB substrate solution (Life technologies) incubated for 15-30 minutes in the dark and stopped with 0,5M sulfuric acid. Assay ODs were measured using the ThermoFisher ELISA Reader (Multiscan Go) and concentrations calculated from standard curves where applicable. Human IFN-γ in the culture supernatant was recorded by using matched pair antibodies (clones NIB 42 and B133.5). Human IL-2 in the culture supernatant was recorded by using matched pair antibodies (B33-2 and 5344.111). Human IL-6 in the culture supernatant was recorded by using IL-6 ELISA Kit (BD Biosciences).

### Cultivation of CAR T Cells

Post-transduction T cells were cultured in RPMI 1640 media with 10% FCS, HEPES buffer, Pen/Strep and 100-300 U/ml IL-2. Culture media was added or exchanged every 3-4 days or when beginning acidification was observed via phenol red color change.

### Cultivation of Tumor Cell Lines

Tumor cell lines were cultured in RPMI 1640 media with 10% (Raji, DOHH-2) or 20% (SU-DHL-10,Oci-Ly1, Oci-Ly19) FCS and Pen/Strep. Cultures were split and media exchanged every 3-4 days or when beginning acidification was observed via phenol red color change. Cultures were checked for mycoplasma via PCR at regular intervals and before in-vivo application.

### Cultivation, Differentiation, and Characterization of Mesenchymal Stem Cells

Mesenchymal stem cells were cultured in Mesenchymal Stem Cell Growth Medium 2. To achieve differentiation, cells were treated with varying concentrations of recombinant TGF-beta 3 (10-20 ng/ml). Cells were then characterized utilizing FACS analysis and anti-CD19 APC-linked (ImmunoTools #21270196) as well as anti-CD248 FITC-linked (Bioss bs-2101R-FITC) antibodies. MACS isolation was achieved using the same antibodies combined with magnetic anti-APC MicroBeads (Miltenyi Biotec #130-090-855) on the autoMACS Pro platform.

### Magnetic-Activated Cell Sorting: Macrophages and T cells

A single-cell suspension from buffy coat derived from healthy donors was prepared using the CD16 MicroBead kit in an autoMACS Pro separator (Miltenyi Biotec). The number of contaminating cells in the isolated monocyte population was less than 2%. Human CD4⁺ and CD8⁺ T cells were isolated using the Pan T cell isolation kit for separation of untouched T cells from peripheral blood mononuclear cells (PBMCs) by depletion of non-T cells (Miltenyi Biotec).

### FACS Analysis of CD80/86 expression on B Cell Lymphoma Cell Lines (DLBCL and CLL) and Healthy B Cells

Tumor cell line expression of antigens and receptors was characterized using FACS analysis utilizing the BD Canto II platform. Cells were quantified and isolated from culture at 5e5-1e6 cells per FACS tube, washed twice each before and after antibody application with 4 ml PBS and incubated with 7-AAD (BD Biosciences 559925) CD5-BV510 (Biolegend 364018) CD19-FITC (Biolegend 392508) CD20-APC/Fire™ 750 (Biolegend 302358) CD80-PE (Biolegend 305208) CD86-APC (Biolegend 374208) at 4° C. for 30 minutes according to manufacturer's instructions.

### Detection of CAR and CAR/CCR Expression Using Flow Cytometry

Transduction efficiency was evaluated via FACS analysis using non-commercial anti-idiotype CD19-CAR (FMC63) antibodies supplied by Miltenyi Biotec and detected with anti-Biotin PE (Miltenyi, Cat #REA746), anti-CTLA4 (CD152) BV421 (BioLegend: Cat #369605), anti-CD3 APC (Miltenyi, Cat #BW264/56) at manufacturer's concentrations. Antibodies were incubated for 30 minutes at 4° C. with two PBS washing steps between primary and secondary antibodies and before analysis. CAR-Transduction efficiency was evaluated in a Lymphocyte/Single Cell/CD3+ gate to confirm successful transduction and normalize CAR-T cell numbers in downstream experiments.

### Details of Xenograft Mouse Trial Studying Efficacy

A total of 28 Rag 2^{-/-} γc^{-/-} mice (Jax mice) between the ages of 100 to 147 days (13 female, 15 male) were intravenously injected with Raji-fLuc cells (5e4 cells per mouse) on day −3. One female mouse was injected intraperitoneally. Tumor engraftment was evaluated on day 3 by injecting D-Luciferin (1.5 mg/mouse) and measuring tumor luminescence in anaesthetized mice in a IVIS200 device (PerkinElmer, Waltham, Massachusetts, USA).

Mice were split into groups receiving intravenously administered 8e6 cells each of the CAR/CCR (6mice), second Generation CAR (7 mice) or untransduced T cells (9 mice) from the same PBMC donor. Six tumor bearing mice remained untreated. All mice were scored daily with luminescence measurements repeated weekly. Mice were sacrificed at predetermined tumor burden or scoring cutoff.

### Details of the Study Using Immunocompetent Mice to Investigate Safety

Mouse splenocytes were genetically engineered to express CAR (2nd) or CAR/CCR constructs, as shown in FIG. 16. CAR and CAR/CCR expression was analyzed by flow cytometry. CAR or CAR/CCR T cells were injected intravenously into the tails of C57bl/6N mice (1×10⁷ per mouse). Blood samples were collected weekly and analyzed for the presence of CD19⁺ cells by flow cytometry using a CD19-specific antibody. Finally, the spleen was removed and also analyzed for the presence of CD19⁺ B cells.

### Immunohistological Analyses

Immunohistological studies of human tissue derived from DLBCL patients (before and upon CAR T cell treatment) were performed on 4-μm-thick sections of the formalin-fixed paraffin-embedded tumour tissue biopsies in whole-section form (University Cologne, Institute for Pathology). For human CD80 detection, slides were primary stained with biotin-labelled polyclonal antibody (5.0 μg/ml) against CD80 (Bioss, Woburn, Massachusetts, USA) and secondary with streptavidin-horseradish peroxidase (500 mU/ml) (Roche, Basel, Suisse).

For human CD86 detection, slides were primary stained with the mouse monoclonal conjugated CD86-specific antibody (2.0 μg/ml), clone SPM600 (Novus Biologicals, Centennial, CO, USA) and secondary with polycional HRP-conjugated mouse IgG1-specific antibody (2.0 μg/ml) (Bioss). Subsequently, both CD80 and CD86 stained sections were additionally incubated with DAB chromogen substrate (Vector Laboratories, Burlingame, CA, USA) and with Haematoxylin (PanReac AppliChem, Cranbury, NJ, USA) for immune-histological analyses, according manufacturer's instructions.

Slides were recorded using the Olympus-UC90 4K microscope (Olympus, Tokyo, Japan) and analysed for CD80 and CD86 expression using ImageJ version 1.53 (National Institutes of Health). Finally, slides were analyzed using an Olympus FV 1000 microscope (Olympus, Tokyo, Japan), and the fluorescence intensity was determined using ImageJ software.

1. Fusion protein comprising:
a) an extracellular domain comprising a polypeptide with specific affinity to CD80 and/or CD86;
b) a transmembrane domain; and
c) an intracellular domain comprising a co-stimulatory polypeptide.

2. Fusion protein according to claim 1, wherein the polypeptide with specific affinity to CD80 and/or CD86 has an amino acid sequence which is at least 80% identical to the amino acid sequence of the extracellular domain of human CTLA-4 (SEQ ID NO: 1) or an amino acid sequence which is at least 80% identical to the amino acid sequence of the extracellular domain of human CD28 (SEQ ID NO: 2), or an amino acid sequence which is at least 80% identical to the amino acid sequence of the CD86-binding domain of the anti-CD86 antibody commonly referred to in the art as clone hu3D1 (SEQ ID NO: 5), preferably wherein the polypeptide with specific affinity to CD80 and/or CD86 has an amino acid sequence which is at least 80% identical to the amino acid sequence of the extracellular domain of human CTLA-4 (SEQ ID NO: 1), and/or wherein the transmembrane domain is suitable for insertion and anchoring of the fusion protein in the cell membrane of a mammalian cell, preferably wherein the transmembrane domain comprises the transmembrane region of one or more of the alpha, beta or zeta chain of the T-cell receptor, CTLA-4, CD28, CD3 epsilon, CD45, CD4, CD5, CD8 (e.g., CD8 alpha, CD8 beta), CD9, CD16, CD22, CD33, CD37, CD64, CD80, CD86, CD134, CD137, CD154, CD200, KIRDS2, 0X40, CD2, CD27, LFA-1 (CDIIa, CD18), ICOS (CD278), 4-1 BB (CD137), GITR, CD40, BAFFR, HVEM (LIGHTR), SLAMF7, NKp80 (KLRF1), CD160, CD19,IL2R beta, IL2R gamma, IL7R .alpha., VLA1, ITGAI, CD49a, ITGA4, IA4, CD49D, ITGA6, VLA-6, CD49f, ITGAD, CDIId, ITGAE, CD103, ITGAL, CDIIa, LFA-1, ITGAM, CDIIb, ITGAX, CDIIc, ITGB1, CD29, ITGB2, CD18, LFA-1, ITGB7, TNFR2, DNAM1 (CD226), SFAMF4 (CD244, 2B4), CD84, CD96 (Tactile), CEACAMI, CRTAM, Ly9(CD229), CD160 (BY55), PSGL1, CD100 (SEMA4D), SLAMF6 (NTB-A, Ly108). SEAM (SLAMF1, CD150, IPO-3), BLAME (SLAMF8), SELPLG (CD162), LTBR, and PAG/Cbp, or an amino acid sequence which is at least 80% identical thereto, preferably wherein the transmembrane domain comprises the transmembrane region of human CTLA-4 (SEQ ID NO: 17), or an amino acid sequence which is at least 80% identical thereto, and/or wherein the intracellular domain comprises the intracellular domain or a costimulatory polypeptide of one or more of human 4-1 BB (CD137; SEQ ID NO: 3), CD3 (SEQ ID NO: 25), CD28, ICOS, OX-40, BTLA, CD27, CD30, GITR, Myd88-CD40, KIR2DS2 and HVEM, or an amino acid sequence which is at least 80% identical thereto, preferably wherein the intracellular domain comprises the intracellular domain or a co-stimulatory polypeptide of human 4-1 BB (CD137; SEQ ID NO: 3), or an amino acid sequence which is at least 80% identical thereto, or wherein the intracellular domain comprises the intracellular domain or a co-stimulatory polypeptide of CD3 (SEQ ID NO: 25), or an amino acid sequence which is at least 80% identical thereto, preferably wherein the intracellular domain comprises the intracellular domain or a costimulatory polypeptide of human 4-1 BB (CD137; SEQ ID NO: 3), or an amino acid sequence which is at least 80% identical thereto.

3. Fusion protein according to claim 1, wherein the extracellular domain comprises a polypeptide comprising the extracellular domain of human CTLA-4 (SEQ ID NO: 1), or an amino acid sequence which is at least 80% identical thereto, and the intracellular domain comprises a co-stimulatory peptide comprising the intracellular domain of human 4-1 BB (CD137; SEQ ID NO: 3), or an amino acid sequence which is at least 80% identical thereto.

4. Nucleic acid molecule encoding the fusion protein of claim 1.

5. Vector comprising the nucleic acid molecule of claim 4.

6. Host cell comprising the nucleic acid molecule of claim 4, preferably wherein the host cell is transduced with the nucleic acid molecule or the vector, or preferably wherein the nucleic acid molecule or the vector of is stably integrated into the genome of the host cell.

7. Host cell stably or transiently expressing a fusion protein according to claim 1.

8. Host cell according to claim 6, wherein the host cell stably or transiently expresses a protein comprising an extracellular domain with specific affinity to CD19 and an intracellular co-stimulatory domain, preferably wherein the extracellular domain with specific affinity to CD19 comprises at least an antigen-binding fragment of an anti-CD19 antibody, more preferably wherein the extracellular domain with specific affinity to CD19 comprises an anti-CD19 scFv, or wherein the host cell stably or transiently expresses a protein comprising an extracellular domain with specific affinity to CD20 and an intracellular co-stimulatory domain, preferably wherein the extracellular domain with specific affinity to CD20comprises at least an antigen-binding fragment of an anti-CD20 antibody, more preferably wherein the extracellular domain with specific affinity to CD20 comprises an anti-CD20 scFv.

9. Host cell according to claim 8, wherein the intracellular costimulatory domain of the fusion protein and/or of the protein comprising an extracellular domain with specific affinity to CD 19 or CD20 comprises an amino acid sequence of the intracellular domain of CD3ζ; (SEQ ID NO: 25), or an amino acid sequence which is at least 80% identical thereto, or an amino acid sequence of the intracellular domain or a co-stimulatory polypeptide of human 4-1 BB (CD137; SEQ ID NO: 3), or an amino acid sequence which is at least 80% identical thereto, preferably wherein the intracellular costimulatory domain of the fusion protein and/or of the protein comprising an extracellular domain with specific affinity to CD 19 or CD20 comprises an amino acid sequence of the intracellular domain of CD3ζ; (SEQ ID NO: 25), or an amino acid sequence which is at least 80% identical thereto.

10. Host cell according to claim 6, wherein the host cell is a human CD8⁺ T cell.

11. Method of providing a host cell of claim 6 comprising:
(1) transducing a host cell with a nucleic acid molecule of or a vector;
(2) cultivating the transduced host cell of in a suitable medium allowing growth of the cell and expression of the fusion protein encoded by said nucleic acid molecule or said vector; and
(3) collecting the host cells from the medium.

12. Host cell obtainable by the method of claim 11.

13. Pharmaceutical composition comprising a fusion protein of claim 1, a nucleic acid molecule, a vector, and/or a host cell.

14. Fusion protein of claim 1, nucleic acid molecule, vector , host cell, or pharmaceutical composition for use as a medicament.

15. Fusion protein of claim 1, nucleic acid molecule, vector, host cell, or pharmaceutical composition for use in the treatment of B cell lymphoma, preferably for the treatment of non-Hodgkin lymphoma selected from the group comprising marginal zone B cell lymphoma (MZL), mucosa-associated lymphatic tissue lymphoma (MALT), small lymphocytic lymphoma/chronic lymphocytic leukemia (SLL/CLL), mantle cell lymphoma (MCL), Burkitt's lymphoma, lymphoplasmacytic lymphoma, Waldenstrom's macroglobulinemia, nodal marginal zone B cell lymphoma (NMZL), splenic marginal zone lymphoma (SMZL), and diffuse large B cell lymphoma (DLBCL), more preferably for the treatment of diffuse large B cell lymphoma (DLBCL).

16. Kit or kit-in-parts comprising a fusion protein of claim 1, nucleic acid molecule, vector, host cell of any-one-of-claims, or pharmaceutical composition, and a container.
